SEQUENCE LISTING (23 sequences)

SEQ ID NO: 1 - protein, 19 aa, Artificial Sequence
  Synthetic Peptide
EEKGQSTSSHSKLSTEGEE

SEQ ID NO: 2 - protein, 4 aa, Artificial Sequence
  Synthetic Peptide
RHKK

SEQ ID NO: 3 - protein, 5 aa, Artificial Sequence
  Synthetic Peptide
KGGAK

SEQ ID NO: 4 - protein, 20 aa, Artificial Sequence
  Synthetic Peptide
EEKGQSTSSHSKLSTEGWEE

SEQ ID NO: 5 - protein, 22 aa, Artificial Sequence
  Synthetic Peptide
EEKGQSTSSHSKLSTEGWAWEE

SEQ ID NO: 6 - protein, 22 aa, Artificial Sequence
  Synthetic Peptide
EEKGQSTSSHSKLSTEGWWFEE

SEQ ID NO: 7 - protein, 22 aa, Artificial Sequence
  Synthetic Peptide
EEKGQSTSSHSKLSTEGWWWEE

SEQ ID NO: 8 - protein, 25 aa, Artificial Sequence
  Synthetic Peptide
ARTKQTARKSTGGKAPRKQLATKAA

SEQ ID NO: 9 - protein, 25 aa, Artificial Sequence
  Synthetic Peptide
ARTKQTARKSTGGKAPRKQLATKAA

SEQ ID NO: 10 - protein, 4 aa, Artificial Sequence
  Synthetic Peptide
RHKK

SEQ ID NO: 11 - protein, 5 aa, Artificial Sequence
  Synthetic Peptide
KGGAK

SEQ ID NO: 12 - protein, 5 aa, Artificial Sequence
  Synthetic Peptide
TARKW

SEQ ID NO: 13 - protein, 9 aa, Artificial Sequence
  Synthetic Peptide
ARTKQTARK

SEQ ID NO: 14 - protein, 14 aa, Artificial Sequence
  Synthetic Peptide
ARTKQTARKSTGGK

SEQ ID NO: 15 - protein, 18 aa, Artificial Sequence
  Synthetic Peptide
ARTKQTARKSTGGKAPRK

SEQ ID NO: 16 - protein, 26 aa, Artificial Sequence
  Synthetic Peptide
ARTKQTARKSTGGKAPRKQLATKAAR

SEQ ID NO: 17 - protein, 25 aa, Artificial Sequence
  Synthetic Peptide
ARTKQTARASTGGKAPRKQLATKAA

SEQ ID NO: 18 - protein, 30 aa, Artificial Sequence
  Synthetic Peptide
NHNHRIRTNPAIVKTENSWSNKAKSICQQQ

SEQ ID NO: 19 - protein, 30 aa, Artificial Sequence
  Synthetic Peptide
VYLYPDIPKEEAFGKYCRPESQEHPEADPG

SEQ ID NO: 20 - protein, 17 aa, Artificial Sequence
  Synthetic Peptide
RRKRAKLIGKYLMGDLL

SEQ ID NO: 21 - protein, 7 aa, Artificial Sequence
  Synthetic Peptide
NNSKFAK

SEQ ID NO: 22 - protein, 7 aa, Artificial Sequence
  Synthetic Peptide
TARKSTG

SEQ ID NO: 23 - protein, 7 aa, Artificial Sequence
  Synthetic Peptide
TARKSTG

(19) United States
(12) Patent Application Publication
(10) Pub. No.: US 2012/0164670 A1
(43) Pub. Date: Jun. 28, 2012

(54) METHODS AND KITS FOR MEASURING ENZYME ACTIVITY

(75) Inventors: Basil Hubbard, Boston, MA (US); David A. Sinclair, Chestnut Hill, MA (US)

(73) Assignee: President and Fellows of Harvard College, Cambridge, MA (US)

(21) Appl. No.: 13/380,108

(22) PCT Filed: Jun. 22, 2010
(86) PCT No.: PCT/US2010/001784

§ 371 Date: Mar. 9, 2012

Related U.S. Application Data
(60) Provisional application No. 61/219,605, filed on Jun. 23, 2009.

(51) Int. Cl. C12Q 1/34; C12Q 1/48

(52) U.S. Cl. 435/15; 435/18

(57) ABSTRACT

The invention relates to methods and kits for measuring enzyme activity, including enzymes that produce nicotinamide.

### RELATED APPLICATIONS

This application claims the benefit under 35 U.S.C. §119(e) of U.S. provisional application Ser. No. 61/219,605, filed on Jun. 23, 2009, the entire disclosure of which is incorporated by reference herein in its entirety.

### FIELD OF INVENTION

The invention relates to methods and compositions for measuring nicotinamide producing reactions.

### BACKGROUND OF INVENTION

Current methods for the measurement of the activity of NAD-cleaving enzymes (such as Sirtuins) are limited. The most widely used method for the measurement of Class III HDAC activity is the BIOMOL assay (Anderson et al., Nature 2003, 423(6936):181-5; Howitz et al., Nature 2003, 425(6954):191-6; Borra et al., J. Biol. Chem. 2005, 280(17):17187-95; Kaeberlein et al., J. Biol. Chem. 2005, 280(17):17038-45). This assay is based upon the deacetylation of a custom fluorophore-tagged substrate (Borra et al., J. Biol. Chem. 2005, 280(17):17187-95; Kaeberlein et al., J. Biol. Chem. 2005, 280(17):17038-45). However, this assay has come under great scrutiny due to potential artifacts resulting from the use of the fluorophore-tag (Borra et al., J. Biol. Chem. 2005, 280(17):17187-95; Kaeberlein et al., J. Biol. Chem. 2005, 280(17):17038-45). Other approaches which have been applied to the measurement of Sirtuin activity include HPLC-based assays and charcoal binding assays using radioactively labeled peptide substrates (Borra et al., J. Biol. Chem. 2005, 280(17):17187-95; Kaeberlein et al., J. Biol. Chem. 2005, 280(17):17038-45). A strategy using mass spectrometry has also been developed for the measurement of SIRT1 activity (Milne et al., Nature 2007, 450(7170):712-6). One of the pitfalls of the current methods being used to measure SIRT1 activity is that they are substrate specific—in most cases requiring a custom-modified peptide substrate (Borra et al., J. Biol. Chem. 2005, 280(17):17187-95; Kaeberlein et al., J. Biol. Chem. 2005, 280(17):17038-45; Milne et al., Nature 2007, 450(7170):712-6).

### SUMMARY OF INVENTION

Disclosed herein are novel assays for measuring the activity of enzymes. Assays described herein can be used to measure the activity of various classes of enzymes including deacetylases, CD38 and related glycohydrolases, PARPs and mono-ADP-ribosyltransferases, PBEF/Nampt and similar enzymes, nicotinamide mononucleotide adenylyltransferase (NMNAT) and nicotinamide ribose kinases (NRK). These methods offer significant advantages over previous techniques for measuring enzymatic activity, including compatibility with a wide range of substrates, safety and cost effectiveness. The assays described herein provide suitable sensitivity and reproducibility, which surprisingly are comparable to known methods such as the BIOMOL assay. Methods described herein can also be used to identify modulators of these classes of enzymes as well as to identify levels of nicotinamide, β-nicotinamide adenine dinucleotide (β-NAD), nicotinamide mononucleotide (NMN) and nicotinamide riboside (NR) in samples. Also described herein are kits for conducting methods associated with the invention.

Aspects of the invention relate to methods for measuring the activity of an enzyme, including combining the enzyme with β-nicotinamide adenine dinucleotide, and optionally an additional substrate, to form a reaction mixture, wherein the enzyme metabolizes β-nicotinamide adenine dinucleotide to produce nicotinamide, adding to the reaction mixture a nicotinamidase in an amount sufficient to produce ammonia from the nicotinamide, and detecting the amount of ammonia produced, wherein the amount of ammonia produced is indicative of the activity of the enzyme. In some embodiments, the enzyme is a Sirtuin such as SIRT1. In certain embodiments the additional substrate is an acetylated polypeptide. In other embodiments, the enzyme is a glycohydrolase such as CD38 or a mono or poly (ADP) ribosyltransferase (mART/PARP). In some embodiments, the nicotinamidase is PNC1 or a homolog thereof.

Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. Absorbance can be read at fixed time intervals or continuously after addition of glutamate dehydrogenase.

Aspects of the invention relate to methods for screening a test molecule for modulation of the activity of an enzyme, the method including, combining the enzyme with β-nicotinamide adenine dinucleotide, and optionally an additional substrate, to form a reaction mixture, wherein the enzyme metabolizes β-nicotinamide adenine dinucleotide to produce nicotinamide, in the presence of the test molecule, adding to the reaction mixture a nicotinamidase in an amount sufficient to produce ammonia from the nicotinamide, detecting the amount of ammonia produced, and comparing the amount of ammonia produced to the amount of ammonia produced in the absence of the test molecule, wherein an increase in the amount of ammonia produced in the presence of the test molecule indicates that the test molecule is an activator of the enzyme, and wherein a decrease in the amount of ammonia produced in the presence of the test molecule indicates that the test molecule is an inhibitor of the enzyme. In some embodiments, the enzyme is a Sirtuin such as SIRT1. In certain embodiments the additional substrate is an acetylated polypeptide. In other embodiments, the enzyme is a glycohydrolase such as CD38 or a mono or poly (ADP) ribosyltransferase (mART/PARP). In some embodiments, the nicotinamidase is PNC1 or a homolog thereof.

Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase.

Aspects of the invention relate to methods for measuring the amount of nicotinamide in a sample, including contacting a sample containing nicotinamide with a nicotinamidase in a reaction mixture, wherein the nicotinamidase produces ammonia from the nicotinamide, and detecting the amount of ammonia produced, wherein the amount of ammonia produced is indicative of the amount of nicotinamide in the sample. Non-limiting examples of samples in which amounts of nicotinamide can be measured include water samples, food samples, tissue samples, cell samples and soil samples.

In some embodiments, the nicotinamidase is PNC1 or a homolog thereof. Detection of ammonia can include reaction of ammonia with o-phthalaldehyde and a reducing agent to produce a fluorescent product, and wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase.

Aspects of the invention relate to methods for measuring the amount of β-nicotinamide adenine dinucleotide in a sample, including contacting a sample containing β-nicotinamide adenine dinucleotide in a reaction mixture with an enzyme, wherein the enzyme metabolizes β-nicotinamide adenine dinucleotide to produce nicotinamide, adding to the reaction mixture a nicotinamidase in an amount sufficient to produce ammonia from the nicotinamide, and detecting the amount of ammonia produced, wherein the amount of ammonia produced is indicative of the amount of β-nicotinamide adenine dinucleotide in the sample. Non-limiting examples of samples in which amounts of β-nicotinamide adenine dinucleotide can be measured include water samples, food samples, tissue samples, cell samples and soil samples. In some embodiments, the enzyme that converts β-nicotinamide adenine dinucleotide to nicotinamide is a glycohydrolase, such as CD38.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase.

Further aspects of the invention relate to methods for measuring the activity of a PBEF/Nampt enzyme, including performing a first reaction by combining the enzyme with a fixed amount of nicotinamide for a first pre-determined time, wherein the enzyme produces nicotinamide mononucleotide from the nicotinamide, and wherein there is a first amount of residual unreacted nicotinamide, and adding to the first reaction a nicotinamidase in an amount and for a time sufficient to produce ammonia from the residual unreacted nicotinamide, and detecting the amount of ammonia produced from the residual unreacted nicotinamide in the first reaction, performing a second reaction by combining the enzyme with a fixed amount of nicotinamide for a second pre-determined time that is less than the first pre-determined time, wherein the enzyme produces nicotinamide mononucleotide from the nicotinamide, and wherein there is a second amount of residual unreacted nicotinamide, and adding to the second reaction a nicotinamidase in an amount and for a time sufficient to produce ammonia from the residual unreacted nicotinamide, and detecting the amount of ammonia produced from the residual unreacted nicotinamide in the second reaction, subtracting the amount of ammonia produced from the residual unreacted nicotinamide in the first reaction from the amount of ammonia produced in the second reaction, wherein the difference between the amount of ammonia produced in the first and second reactions is indicative of the activity of the enzyme.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. In some embodiments, the second pre-determined time is zero.

Further aspects of the invention relate to methods for measuring the activity of an enzyme, wherein the enzyme has nicotinamide mononucleotide adenylyltransferase activity, including combining the enzyme with nicotinamide mononucleotide to form a reaction mixture, wherein the enzyme produces β-nicotinamide adenine dinucleotide from the nicotinamide mononucleotide, adding to the reaction mixture a glycohydrolase in an amount sufficient to produce nicotinamide from the β-nicotinamide adenine dinucleotide, adding to the reaction mixture a nicotinamidase in an amount sufficient to produce ammonia from the nicotinamide, and detecting the amount of ammonia produced, wherein the amount of ammonia produced is indicative of the activity of the enzyme. In some embodiments the glycohydrolase is CD38.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. In some embodiments, the second pre-determined time is zero.

Aspects of the invention relate to methods for measuring the activity of an enzyme, wherein the enzyme has nicotinamide riboside kinase activity, including combining the enzyme with nicotinamide riboside to form a reaction mixture, wherein the enzyme produces nicotinamide mononucleotide from the nicotinamide riboside, adding to the reaction mixture a nicotinamide mononucleotide adenylyltransferase in an amount sufficient to produce β-nicotinamide adenine dinucleotide from the nicotinamide mononucleotide, adding to the reaction mixture a glycohydrolase in an amount sufficient to produce nicotinamide from the β-nicotinamide adenine dinucleotide, adding to the reaction mixture a nicotinamidase in an amount sufficient to produce ammonia from the nicotinamide, and detecting the amount of ammonia produced, wherein the amount of ammonia produced is indicative of the activity of the enzyme. In some embodiments, the glycohydrolase is CD38.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. In some embodiments, the second pre-determined time is zero.

Aspects of the invention relate to methods for measuring the amount of nicotinamide mononucleotide in a sample, including contacting a sample containing nicotinamide mononucleotide with a nicotinamide mononucleotide adenylyltransferase in a reaction mixture, wherein the nicotinamide mononucleotide adenylyltransferase is present in the reaction mixture in an amount sufficient to produce β-nicotinamide adenine dinucleotide, adding to the reaction mixture a glycohydrolase in an amount sufficient to produce nicotinamide from the β-nicotinamide adenine dinucleotide, adding to the reaction mixture a nicotinamidase in an amount sufficient to produce ammonia from the nicotinamide, and detecting the amount of ammonia produced, wherein the amount of ammonia produced is indicative of the amount of nicotinamide mononucleotide in the sample. In some embodiments, the glycohydrolase is CD38.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. In some embodiments, the second pre-determined time is zero.

Further aspects of the invention relate to methods for measuring the amount of nicotinamide riboside in a sample, including contacting a sample containing nicotinamide riboside with a nicotinamide riboside kinase in a reaction mixture, wherein the nicotinamide riboside kinase is present in the reaction mixture in an amount sufficient to produce nicotinamide mononucleotide from the nicotinamide riboside, adding to the reaction mixture a nicotinamide mononucleotide adenylyltransferase in an amount sufficient to produce β-nicotinamide adenine dinucleotide from the nicotinamide mononucleotide, adding to the reaction mixture a glycohydrolase in an amount sufficient to produce nicotinamide from the β-nicotinamide adenine dinucleotide, adding to the reaction mixture a nicotinamidase in an amount sufficient to produce ammonia from the nicotinamide, and detecting the amount of ammonia produced, wherein the amount of ammonia produced is indicative of the amount of nicotinamide riboside in the sample. In some embodiments, the glycohydrolase is CD38.

In some embodiments, the nicotinamidase is PNC1 or a homolog thereof. Detection of ammonia can include reaction of ammonia with o-phthalaldehyde and a reducing agent to produce a fluorescent product, wherein the fluorescent product is detected. In some embodiments, the reducing agent is DTT, β-mercaptoethanol, thioglycolic acid or sodium hydrosulfite. In some embodiments, the enzymatic reaction is terminated prior to addition of the nicotinamidase.

Detection of ammonia can also involve addition of a-Ketoglutarate and NADPH and taking a first absorbance measurement, followed by addition of glutamate dehydrogenase and taking a second absorbance measurement, wherein the difference in absorbance between the first and second absorbance measurements is indicative of the amount of ammonia present. In some embodiments, a-Ketoglutarate and NADPH are added simultaneously with the nicotinamidase, while in other embodiments, a-Ketoglutarate and NADPH are added after the nicotinamidase. In some embodiments, the second pre-determined time is zero.

Further aspects of the invention relate to kits. In some embodiments, a kit for measuring the activity of an enzyme can include a nicotinamidase protein, O-phthalaldehyde, and instructions for use of components of the kit for measuring the activity of an enzyme. In other embodiments, a kit for measuring the activity of an enzyme can include a-Ketoglutarate, NADPH, a nicotinamidase protein, glutamate dehydrogenase, and instructions for use of components of the kit for measuring the activity of an enzyme that produces nicotinamide.

In some embodiments, a kit for screening a test molecule for modulation of the activity of an enzyme can include an enzyme, optionally a substrate molecule, β-nicotinamide adenine dinucleotide, a nicotinamidase protein, O-phthalaldehyde, and instructions for use of components of the kit for screening a test molecule for modulation of the activity of an enzyme. In other embodiments, a kit for screening a test molecule for modulation of the activity of an enzyme includes an enzyme, optionally a substrate molecule, β-nicotinamide adenine dinucleotide, a-Ketoglutarate, NADPH, a nicotinamidase protein, glutamate dehydrogenase, and instructions for use of components of the kit for screening a test molecule for modulation of the activity of an enzyme.

In some embodiments, a kit for measuring the amount of nicotinamide in a sample includes a nicotinamidase protein, O-phthalaldehyde, and instructions for use of components of the kit for measuring the amount of nicotinamide in the sample. In other embodiments, a kit for measuring the amount of nicotinamide in a sample can include a-Ketoglutarate, NADPH, a nicotinamidase protein, glutamate dehydrogenase and instructions for use of components of the kit for measuring the amount of nicotinamide in the sample.

In some embodiments, a kit for measuring the amount of β-nicotinamide adenine dinucleotide in a sample can include a glycohydrolase enzyme, a nicotinamidase protein, O-phthalaldehyde, and instructions for use of components of the kit for measuring the amount of β-nicotinamide adenine dinucleotide in the sample. In other embodiments, a kit for measuring the amount of β-nicotinamide adenine dinucleotide in a sample can include a glycohydrolase enzyme, a-Ketoglutarate, NADPH, a nicotinamidase protein, glutamate dehydrogenase and instructions for use of components of the kit for measuring the amount of nicotinamide in the sample. In some embodiments, any of the kits described herein can further comprise a positive control.

Methods and products described herein can also be applied to mechanistic studies of small molecule activators or inhibitors of any of the enzymes described herein or other enzymes having the same or similar substrate specificity, screening of substrates to characterize substrate-specific activators or inhibitors of any of the enzymes described herein or other enzymes having the same or similar substrate specificity, identification and characterization of endogenous protein activators or inhibitors of any of the enzymes described herein or other enzymes having the same or similar substrate specificity, and determining substrate preference and/or kinetic parameters for substrates of any of the enzymes described herein or other enzymes having the same or similar substrate specificity.

These and other aspects of the invention, as well as various embodiments thereof, will become more apparent in reference to the drawings and detailed description of the invention.

### BRIEF DESCRIPTION OF DRAWINGS

The accompanying drawings are not intended to be drawn to scale. In the drawings, each identical or nearly identical component that is illustrated in various figures is represented by a like numeral. For purposes of clarity, not every component may be labeled in every drawing. In the drawings:

FIG. 1 presents a schematic and a graph outlining a Nicotinamide Assay. FIG. 1A presents a schematic showing a SIRT1 enzymatic deacetylation reaction carried out in the presence of an acetylated substrate and β-NAD. The schematic indicates addition of PNC1, a-Ketoglutarate, NADPH and glutamate dehydrogenase to the reaction. The assay can be run at fixed time points or continuously without inactivation of SIRT1. FIG. 1B presents a graph depicting a standard curve for nicotinamide conversion. Increasing amounts of nicotinamide were subject to the coupled PNC1-GDH reaction, and the reduction in absorbance at 340 nm was monitored using a spectrophotometer. Experiments were performed in triplicate; mean+/−SD is shown.

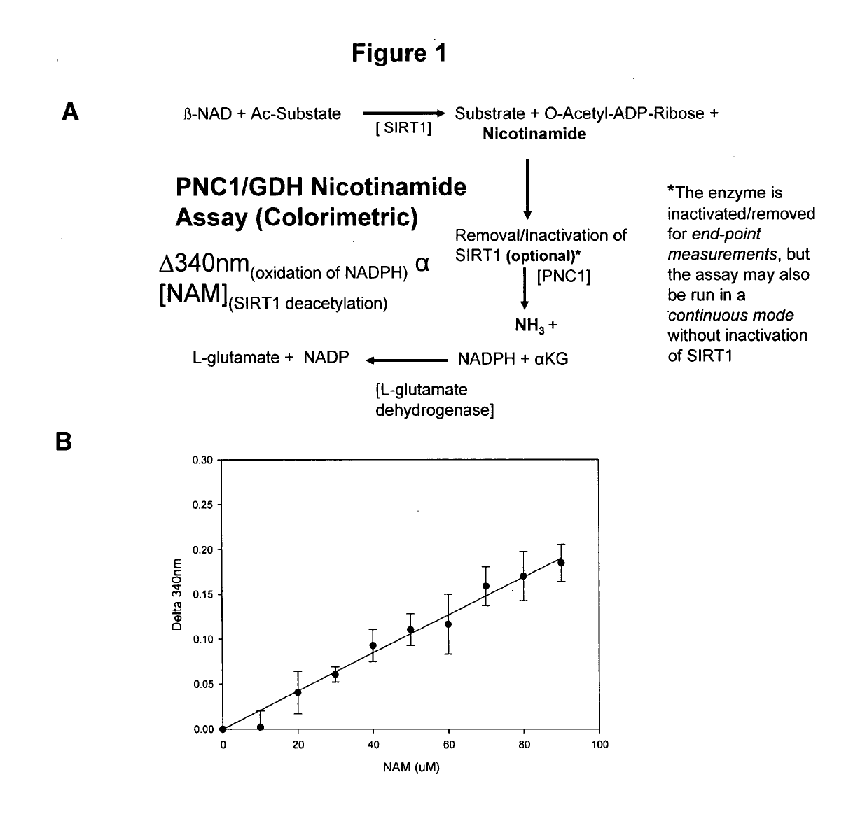

FIG. 2 presents standard curve graphs outlining the ability of the PNC1-GDH method to accurately quantify ammonia and nicotinamide levels. FIG. 2A depicts a standard curve for ammonia using the PNC1-GDH assay. The linear fit equation and coefficient of correlation are displayed on the graph. FIG. 2B depicts a standard curve for nicotinamide using the PNC1-GDH assay using several different PNC1-incubation times (all are linear). In both cases, means+/−standard deviation of three replicates is shown.

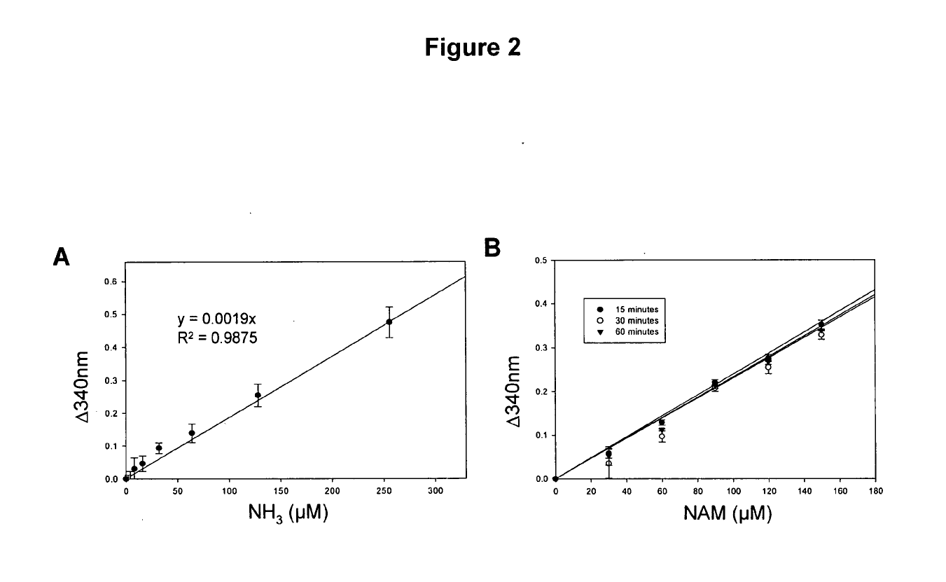

FIG. 3 presents a graph depicting a time course of SIRT1 deacetylation. SIRT1 deacetylation was monitored over several hours using the Nicotinamide Assay depicted in FIG. 1. The amount of nicotinamide produced was proportional to the length of time the SIRT1 reaction was allowed to proceed. Experiments were performed in triplicate; mean+/−SD is shown.

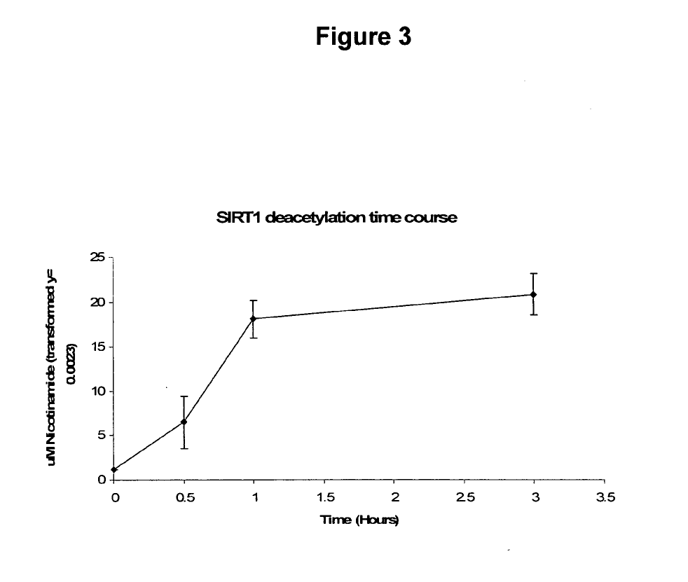

FIG. 4 presents a graph depicting determination of NAD Km value for SIRT1 using the Nicotinamide Assay depicted in FIG. 1. The experiment yields the correct Km for NAD (−80-100 uM). Experiments were performed in triplicate; mean+/−SD is shown.

FIG. 5 presents a graph depicting a test of SIRT1-specific chemical inhibitors. SIRT1 deacetylation reactions were carried out in the absence or presence of various doses of the SIRT1 inhibitor EX-527. Subsequently, the amount of nicotinamide produced after 3 hours was monitored using the Nicotinamide Assay depicted in FIG. 1.

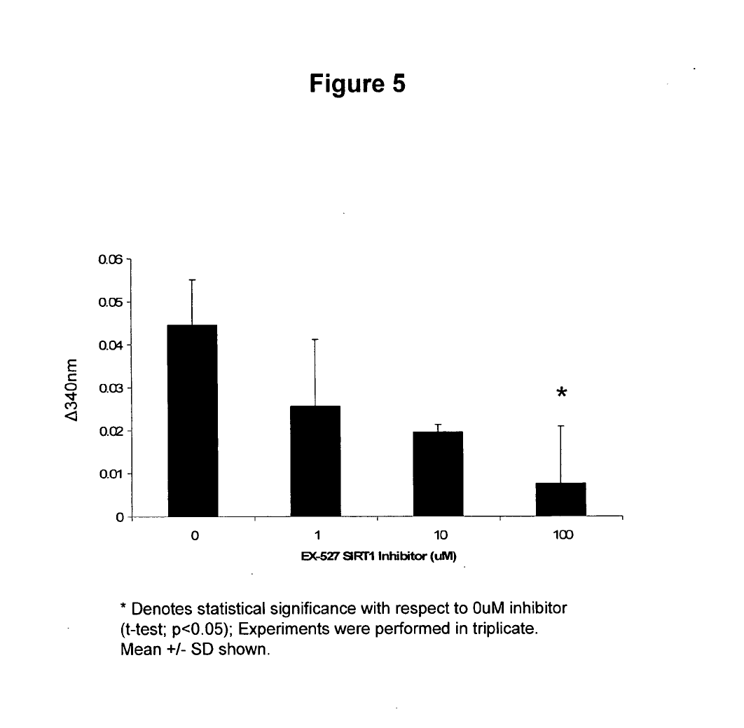

FIG. 6 presents a schematic and a graph outlining a fluorescent Nicotinamide Assay. FIG. 6A presents a schematic showing a SIRT1 enzymatic deacetylation reaction carried out in the presence of an acetylated substrate and β-NAD. The schematic indicates addition of PNC1, O-phthalaldehyde and DTT to the reaction. FIG. 6B presents a graph depicting a standard curve for nicotinamide conversion. Increasing amounts of nicotinamide were subject to the coupled PNC1-OPT reaction, and the resulting fluorescence was measured (excitation 420 nm and emission at 460 nm). Fluorescence is expressed in relative fluorescence units (rFU). Experiments were performed in triplicate; mean+/−SD is shown.

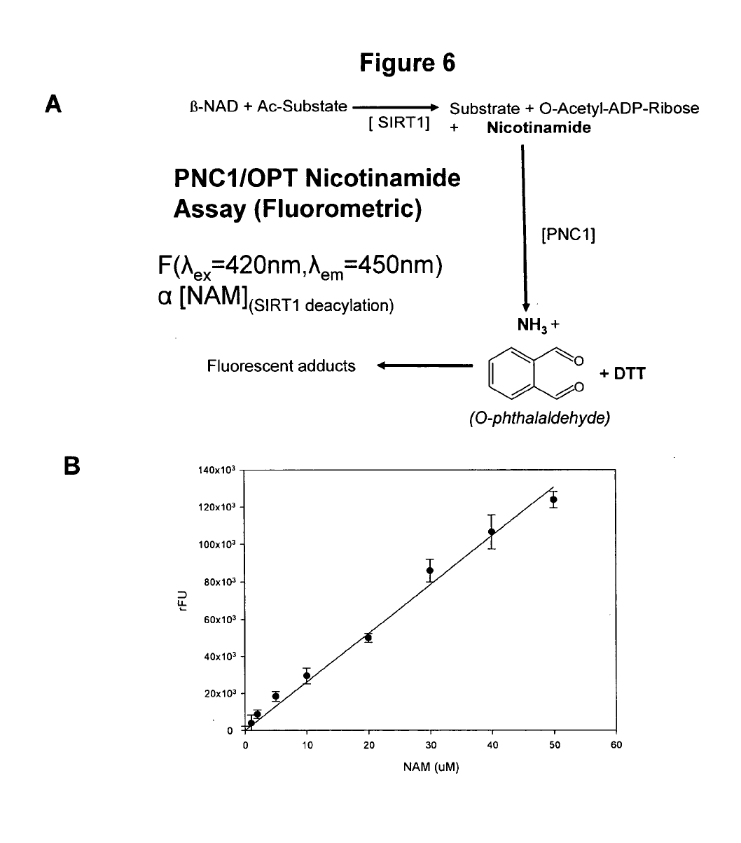

FIG. 7 displays standard curve graphs outlining the ability of the PNC1-OPT method to accurately quantify ammonia and nicotinamide levels. FIG. 7A depicts a standard curve for ammonia using the PNC1-OPT assay. The linear fit equation and coefficient of correlation are displayed on the graph. FIG. 7B depicts a standard curve for nicotinamide using the PNC1-OPT. The linear fit equation and coefficient of correlation are displayed on the graph. FIG. 7C represents a PNC1 saturation experiment in which the indicated amounts of PNC1 were used in the PNC1-OPT assay to detect the products of a SIRT1 reaction (H3K9 peptide at 100 μM, β-NAD at 200 μM). Means+/−standard deviation of three replicates are shown.

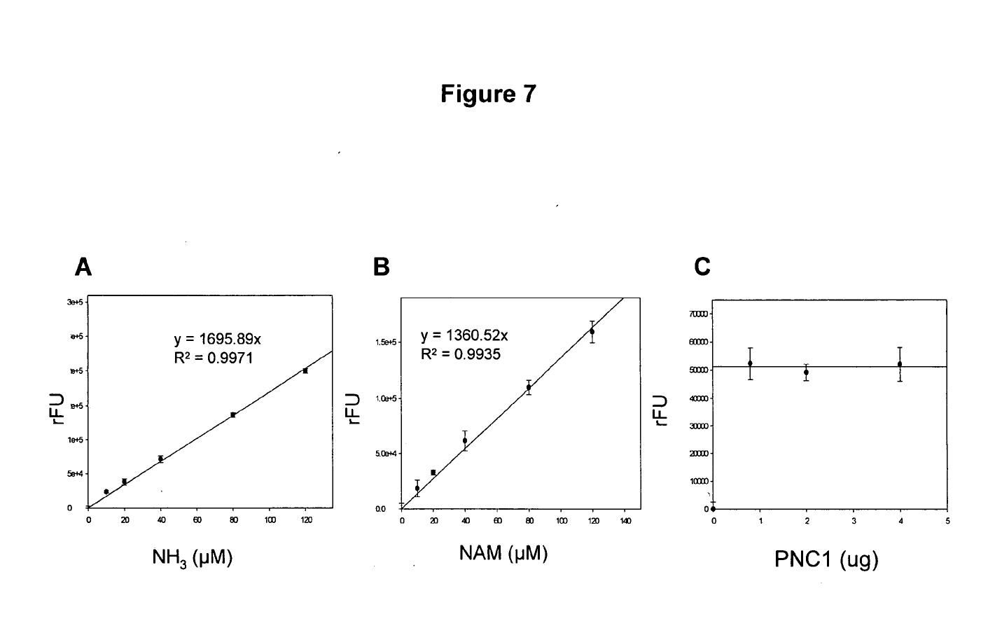

FIG. 8 presents a series of experiments outlining the functionality of the PNC1-OPT nicotinamide assay using the commercially available Fleur de Lys peptide (BIOMOL) as the substrate. FIG. 8A depicts a time course for deacetylation of the Fleur de Lys peptide. The relative fluorescence was proportional to the amount of time the reaction was allowed to proceed for. FIG. 8B displays a titration of SIRT1 enzyme activity (using increasing amount of SIRT1 enzyme) with 200 uM β-NAD and 100 uM of the Fleur de Lys substrate. The fluorescent signal obtained was proportional to the amount of SIRT1 enzyme added (and linear up to a range of approximately 5 ug). FIG. 8C presents a graph depicting determination of Fleur de Lys peptide Km value for SIRT1 using the Nicotinamide Assay depicted in FIG. 6. The experiment yields the correct Km for Fleur de Lys (˜130.2 uM). FIG. 8D presents a graph depicting determination of NAD Km value for SIRT1 using the Nicotinamide Assay depicted in FIG. 6. The experiment yields the correct Km for NAD (˜200 uM). Experiments were performed in triplicate; mean+/−SD is shown.

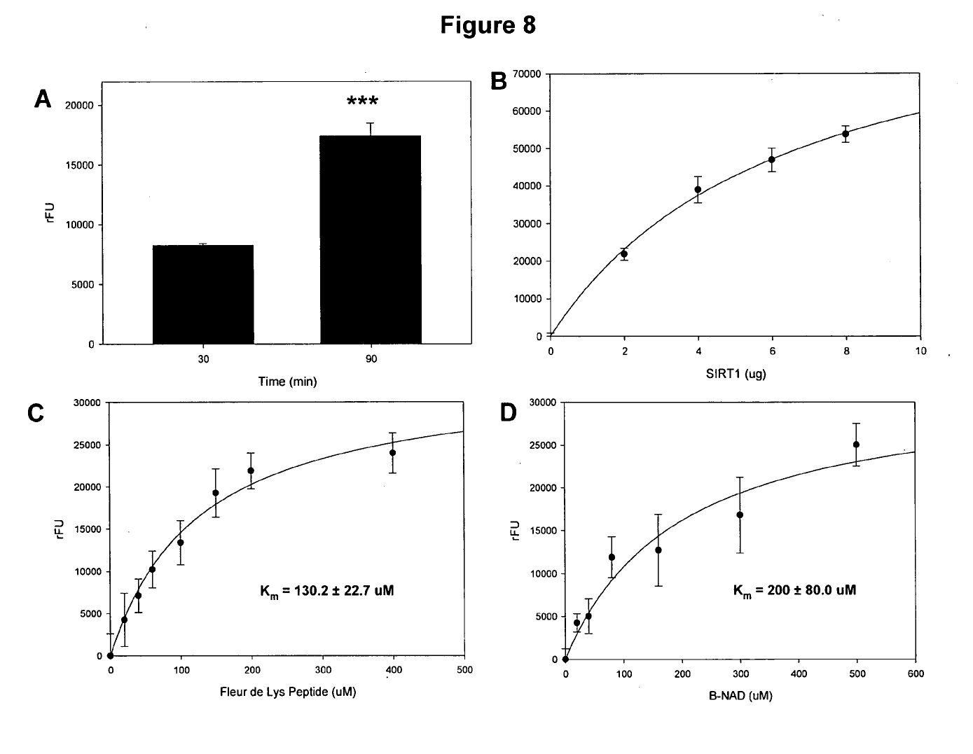

FIG. 9 represents an analysis of enzyme activity versus enzyme concentration and time using the PNC1-OPT assay. FIG. 9A shows the linearity of activity with respect to time for various concentrations of SIRT1 enzyme. Linear fit equations for each enzyme concentration are included. FIG. 9B shows the relationship between the slopes of these equations versus enzyme concentration; there is a linear dependence of activity with respect to enzyme concentration (over several time points). Means+/−standard deviation of three replicates are shown.

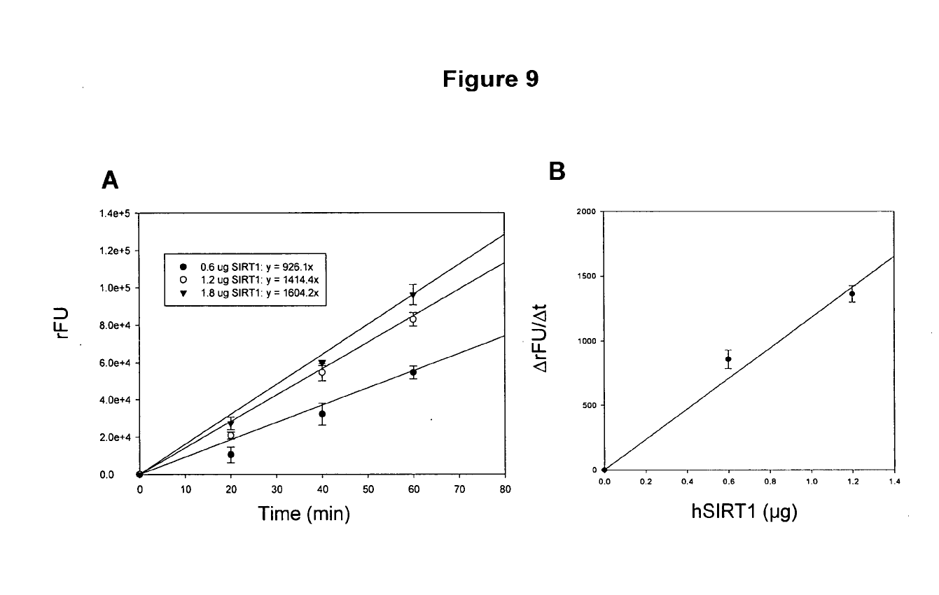

FIG. 10 presents a graph depicting several tests of SIRT1-specific chemical inhibitors and activators using the assay described in FIG. 6 with the Fleur de Lys substrate. FIG. 10A shows that 500 uM Splitomicin has a weak inhibitory effect on SIRT1 deacetylation. FIG. 10B displays a graph showing that SRT91211 shows a potent inhibitory effect on SIRT1 deacetylation in the PNC1-OPT Nicotinamide assay, consistent with literature. FIG. 10C presents a graph showing activation of SIRT1 deacetylase activity on the Fleur de Lys substrate with 100 uM Resveratrol. This experiment was performed in the presence of 75 uM β-NAD and 25 uM Fleur de Lys substrate. Experiments were performed in triplicate; mean+/−SD is shown.

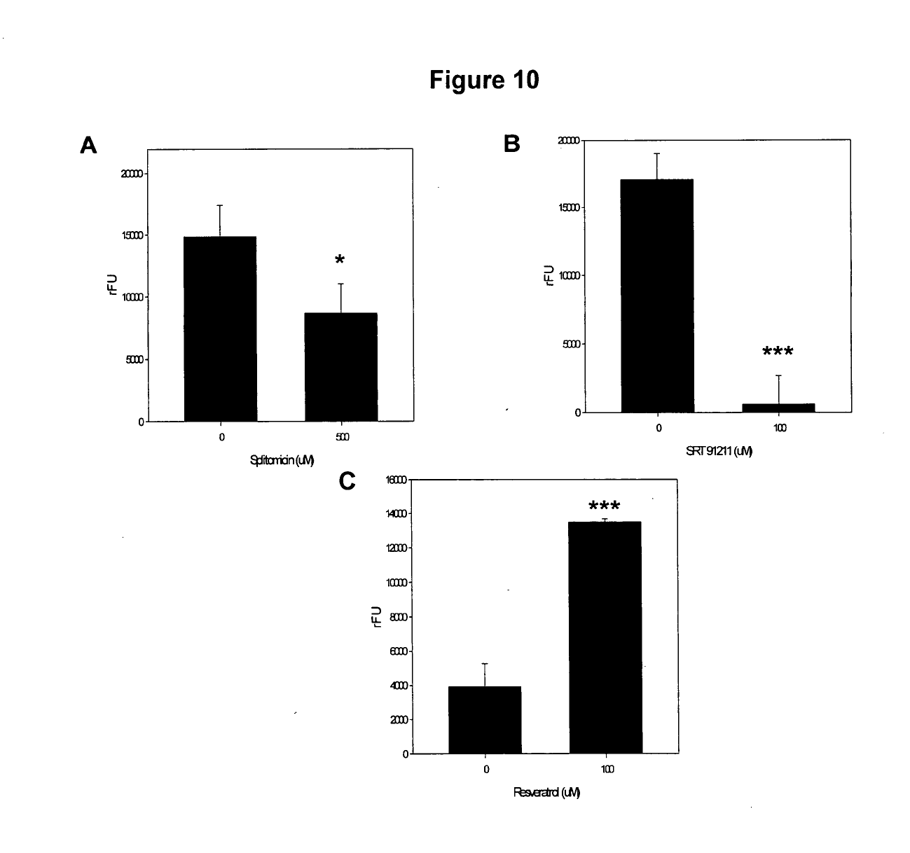

FIG. 11 presents a list of synthetic native and non-native peptide substrates which were produced for use with the PNC1-OPT reaction (FIG. 6). The ability of the PNC1-OPT nicotinamide assay to use these custom substrates is demonstrated in FIG. 12. The peptides depicted in FIG. 11 correspond to SEQ ID NOs:1-9 respectively.

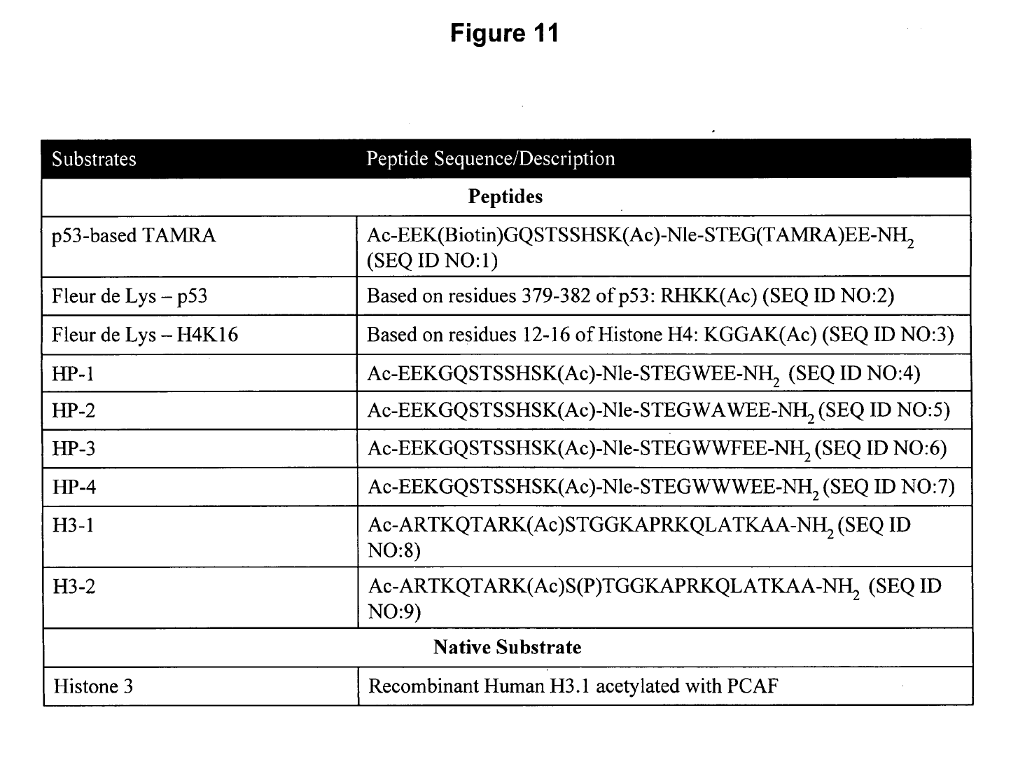

FIG. 12 presents several graphs demonstrating the use of the PNC1-OPT nicotinamide assay with several custom substrates (described in detail in FIG. 11). FIG. 12A shows the relative deacetylation rates of two native peptide substrates (TAMRA (no tag), H3K9) in comparison to two fluorophore tagged substrates (FdL-p53, FdL-H4K16). FIG. 12B presents a graph comparing the deacetylation rate of phosphorylated H3K9(ac) to its non-phosphorylated equivalent. FIG. 12C depicts a graph showing the deacetylation of various hydrophobic patch peptides (described in FIG. 11). FIG. 12D presents a graph comparing the effects of 100 uM Resveratrol on a native peptide substrate (H3K9ac) in comparison to the FdL-p53 peptide. Experiments were performed in triplicate; mean+/−SD is shown.

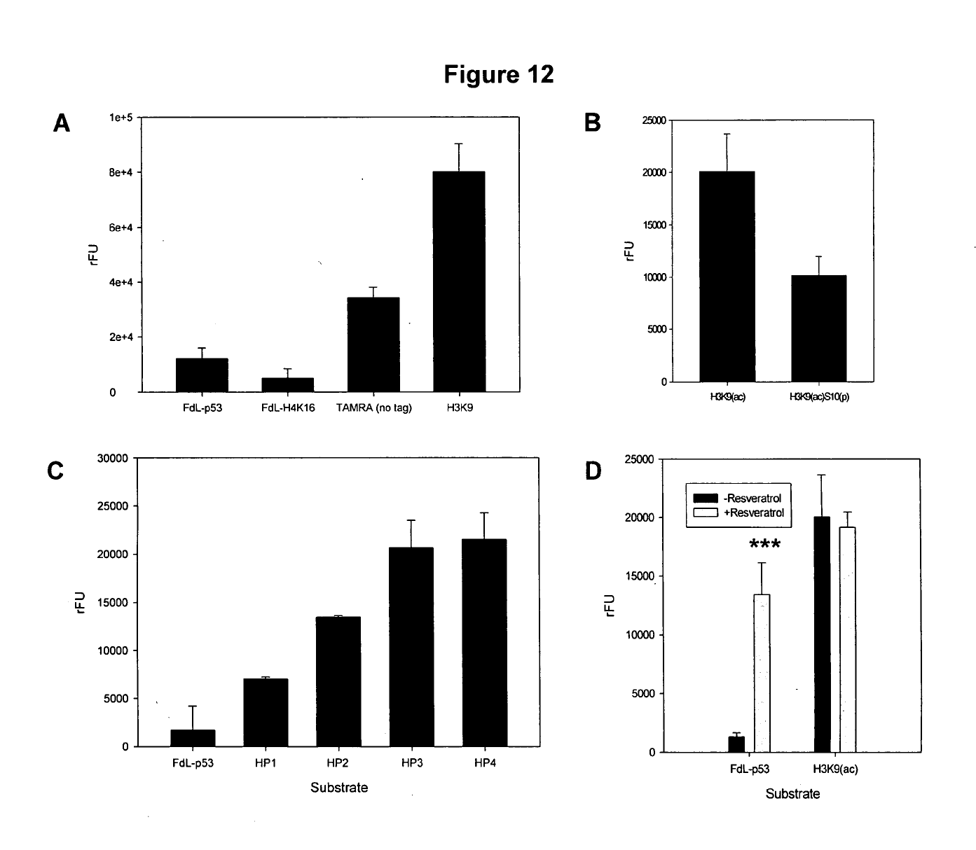

FIG. 13 presents competition assays between several of the peptides described in FIG. 11 with the Fleur de lys-p53 peptide using the BIOMOL assay. FIG. 13A presents the results of a BIOMOL assay using 100 uM β-NAD and 50 uM Fleur de lys-p53 peptide in the absence (−) or presence of 50 uM of a competing acetylated peptide (H3-1, H3-2). FIG. 13B presents the results of a BIOMOL assay using 100 uM β-NAD and 50 uM Fleur de lys-p53 peptide in the absence (−) or presence of 50 uM of a competing acetylated peptide (HP1, HP2, HP3, HP4). Peptide descriptions are presented in FIG. 11. These results are in agreement with the results obtained using the PNC1-OPT Nicotinamide assay in FIG. 12. Experiments were performed in triplicate; mean+/−SD is shown.

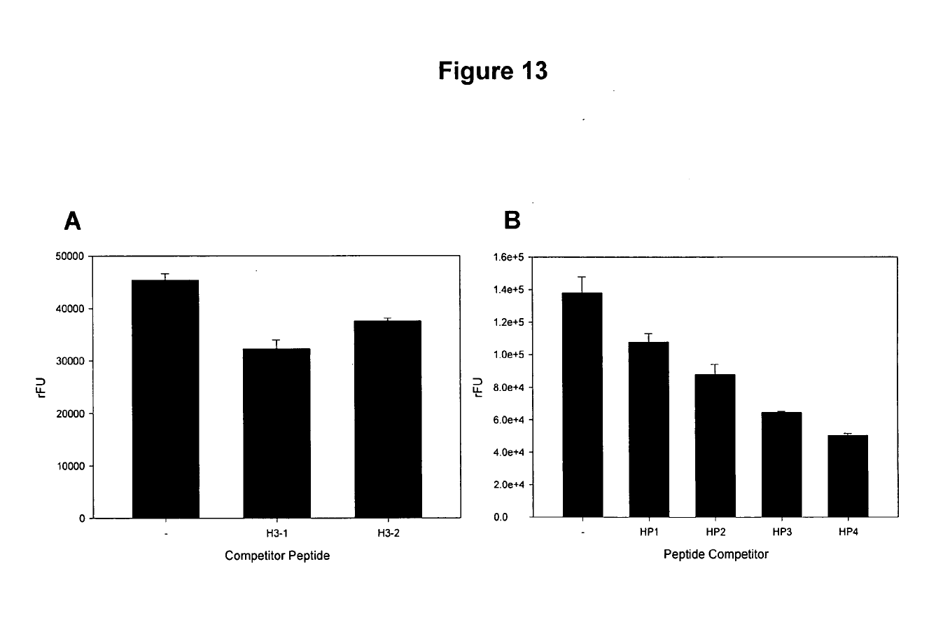

FIG. 14 presents a graph displaying the measurement of SIRT1 deacetylase activity on a whole protein native substrate. Recombinant Histone H3 was acetylated in vitro using PCAF, and used as the substrate in a downstream PNC1-OPT assay. The results display the ability of the assay to detect NAD-dependent deacetylase activity using this native, whole-protein substrate. Means+/−standard deviation of three replicates are shown.

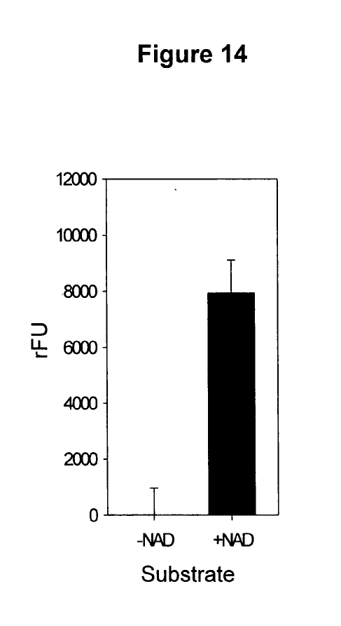

FIG. 15 presents two graphs displaying the Km determination for two substrate peptides, H3-1 (non-phosphorylated H3 peptide) and H3-2 (phosphorylated H3 peptide) using the PNC1-OPT assay. FIG. 15A represents the Km determination for substrate H3-1. FIG. 15B displays the Km determination for substrate H3-2. Any background fluorescence due to p-NAD or the peptide substrate were corrected for by subtracting out appropriate blank controls. For both assays, β-NAD was used at a saturating concentration of 1.5 mM. Means+/−standard deviation of three replicates are shown.

FIG. 16 presents two tables displaying the sequences of several peptides synthesized for use with PNC1-based assays and kinetic parameters for selected substrates determined using the PNC1-OPT assay. The peptide sequences in FIG. 16A correspond to SEQ ID NOs: 10-17, 8, 9, 4-7, 18-20, 12, 21 and 22 respectively. Any background fluorescence due to f3-NAD or the peptide substrate were corrected for by subtracting out appropriate blank controls. An asterisk indicates that DTT was added to the deacetylation reaction in order to prevent disulfide bond formation between substrate molecules. Means+/−standard deviation of two separate Km determinations are presented.

FIG. 17 depicts the primary data plot of a matrix experiment using the PNC1-OPT assay in which SIRT1 deacetylase activity was measured under varied conditions of substrate and β-NAD (concentrations indicated). FIG. 17A displays Michaelis-Menten curves for f3-NAD at various fixed concentrations of H3K9(L) peptide substrate. FIG. 17B displays Michaelis-Menten curves for H3K9(L) peptide at various fixed concentrations of β-NAD. Any background fluorescence due to β-NAD or the peptide substrate were corrected for by subtracting out appropriate blank controls.

FIG. 18 displays secondary analysis plots (data from FIG. 17) of Vmax and Vmax/K vs substrate and β-NAD elucidating the enzymatic mechanism of SIRT1. FIG. 17A displays the variation of apparent Vmax with respect to H3K9 peptide concentration. FIG. 17B displays the variation of apparent Vmax with results to β-NAD. FIG. 17C presents the variation of apparent Vmax/apparent Km with respect to H3K9 peptide concentration. FIG. 17D presents the variation of apparent Vmax/apparent Km with respect to β-NAD. Based on the shapes of these graphs, it is evident that the SIRT1 enzymatic reaction proceeds via a sequential mechanism (random or ordered; not ping-pong).

FIG. 19 presents the results of a mechanistic analysis of the SIRT1 inhibitor Adenosine diphosphate ribose (ADPr) using the PNC1-OPT assay. FIG. 19A displays Michaelis-Menten curves for β-NAD at various fixed concentrations of ADPr. FIG. 19B displays Michaelis-Menten curves for H3K9 peptide at several fixed concentrations of ADPr. Appropriate controls were used.

FIG. 20 presents the secondary analysis plots (data from FIG. 19) of Vmax and Vmax/K vs [ADPr] for both NAD and H3K9, elucidating the mechanism of SIRT1 inhibition by ADPr. FIG. 20A displays the effect of inhibitor on apparent Vmax for β-NAD. FIG. 20B displays the effect of inhibitor on apparent Vmax/K for β-NAD. FIG. 20C displays the effect of inhibitor on Vmax for H3K9. FIG. 20D displays the effect of inhibitor on Vmax/K for H3K9. The overall results of this analysis suggest ADPr inhibits SIRT1 by acting predominately as a competitive inhibitor of β-NAD, with the possibility of an additional non-competitive component.

FIG. 21 presents results showing the effect of the endogenous SIRT1 protein inhibitor DBC1 on SIRT1 activity using the PNC1-OPT assay. FIG. 21A shows the degree of SIRT1 inhibition by DBC1 using two different native peptides (both used at a final concentration of 25 μM). FIG. 21B displays a dose-titration experiment of DBC1 versus SIRT1 activity using an H3K9-L peptide substrate. Reactions in the absence of NAD were used to correct for background fluorescence. DBC1 concentration inversely correlates with SIRT1 activity. Means+/−standard deviation of two separate Km determinations are presented.

FIG. 22 presents graphs evaluating the effects of several known polyphenol SIRT1 activators using AMC-tagged substrates with the PNC1-OPT assay. FIG. 22A displays the effects of Piceatannol on the deacetylation of an H3K9-AMC peptide by SIRT1. FIG. 22B depicts the results of a dose titration of Resveratrol versus enzymatic activity of SIRT1 using an H3K9-AMC substrate. FIG. 22C displays the effects of Resveratrol on several custom substrates in which the AMC moiety is located at various distances from the acetylated lysine residue (5, 9, and 17 amino acids away). In all cases, the β-NAD concentration used was 75 μM and the AMC-peptide concentration was 25 μM. Means+/−standard deviation of three trials are presented.

FIG. 23 presents graphs evaluating the effects of Resveratrol and an additional Sirtuin Activating compound on several native (non-tagged) and tagged peptide substrates with the PNC1-OPT assay. FIG. 23A-FIG. 23C display the effects of Resveratrol on several custom and commercially available fluorophore-tagged and non-tagged substrates. In all cases, the β-NAD concentration used was 75 μM and the AMC-peptide concentration was 25 μM. FIG. 23D presents data showing that activation of a non-fluorophore tagged substrate is possible using a SIRT1 activator. Appropriate controls and blanks were performed. Means+/−standard deviation of three trials are presented.

FIG. 24 presents the results of an activity analysis of several SIRT1 mutations/deletions found in certain cancer cell lines using the PNC1-OPT assay. Recombinant proteins corresponding to the various mutations/deletions were produced and their activity was assayed in comparison to the wild-type SIRT1 enzyme. 200 μMβ-NAD was present in each reaction and 100 μM H3K9-S (TARK(ac)STG)) (SEQ ID NO:23) was used as substrate. Means+/−standard deviation of three trials are presented.

FIG. 25 presents a schematic outlining an assay for measuring nicotinamide levels in a sample.

FIG. 26 presents a schematic outlining an assay for measuring the activity of a glycohydrolase such as CD38.

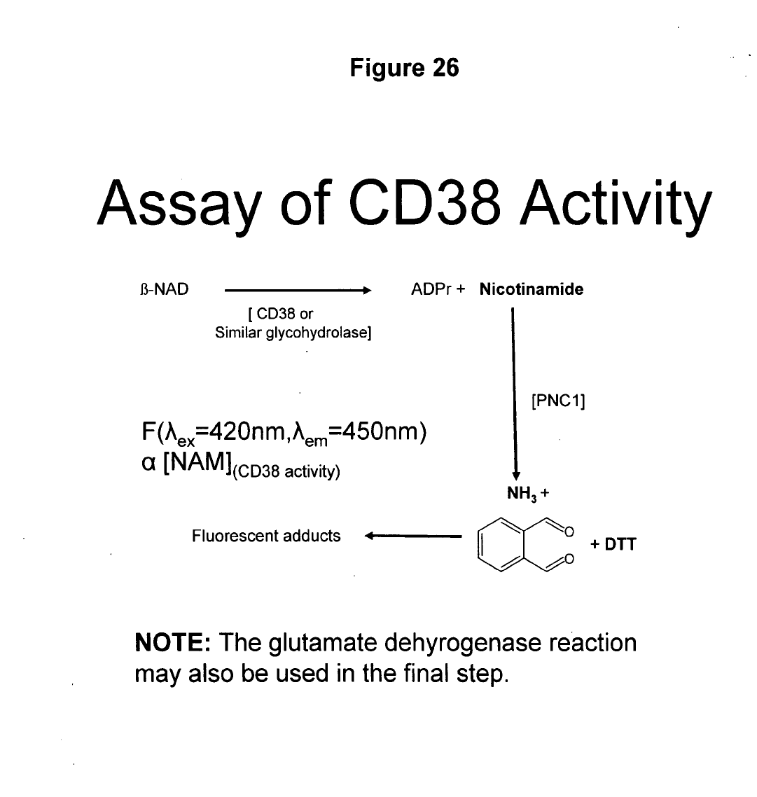

FIG. 27 presents a graph revealing results obtained using the assay depicted in FIG. 26. The activity of CD38 was assayed with 100 μm β-NAD, in the presence of various inhibitors. Measurements were performed in triplicate; means of the data are presented.

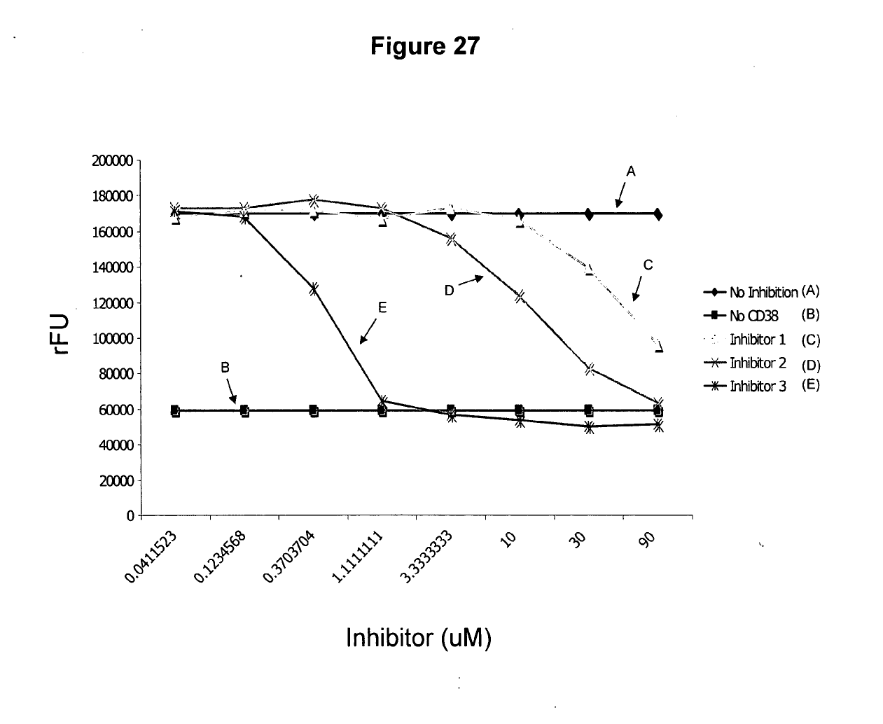

FIG. 28 presents a schematic outlining an assay for measuring the activity of a mono or poly(ADP) ribosyltransferase.

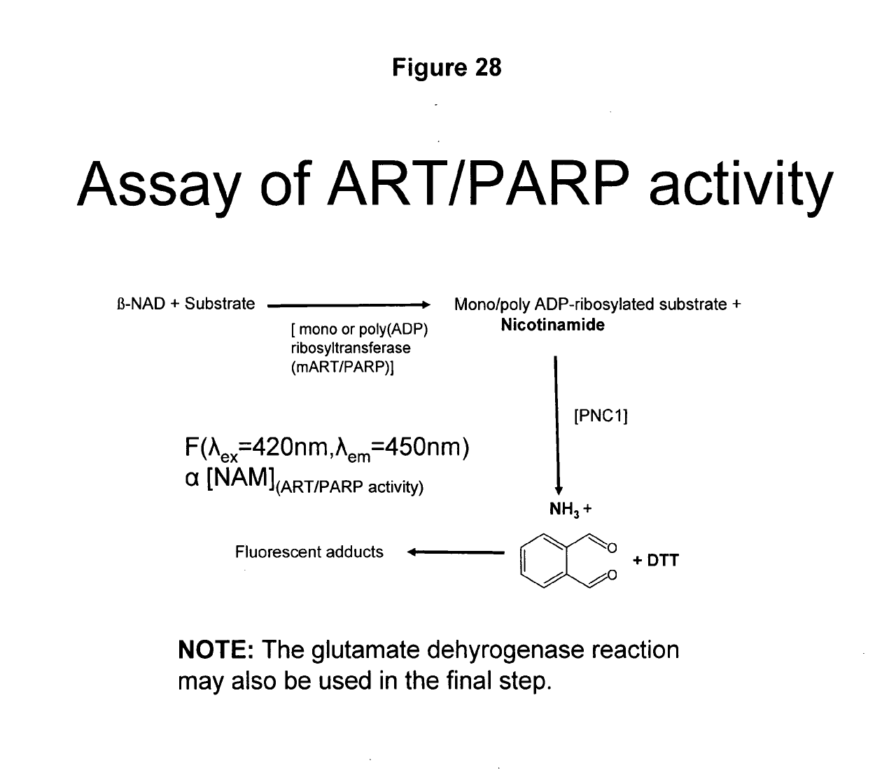

FIG. 29 presents a schematic outlining an assay for measuring the amount of β-NAD in a sample.

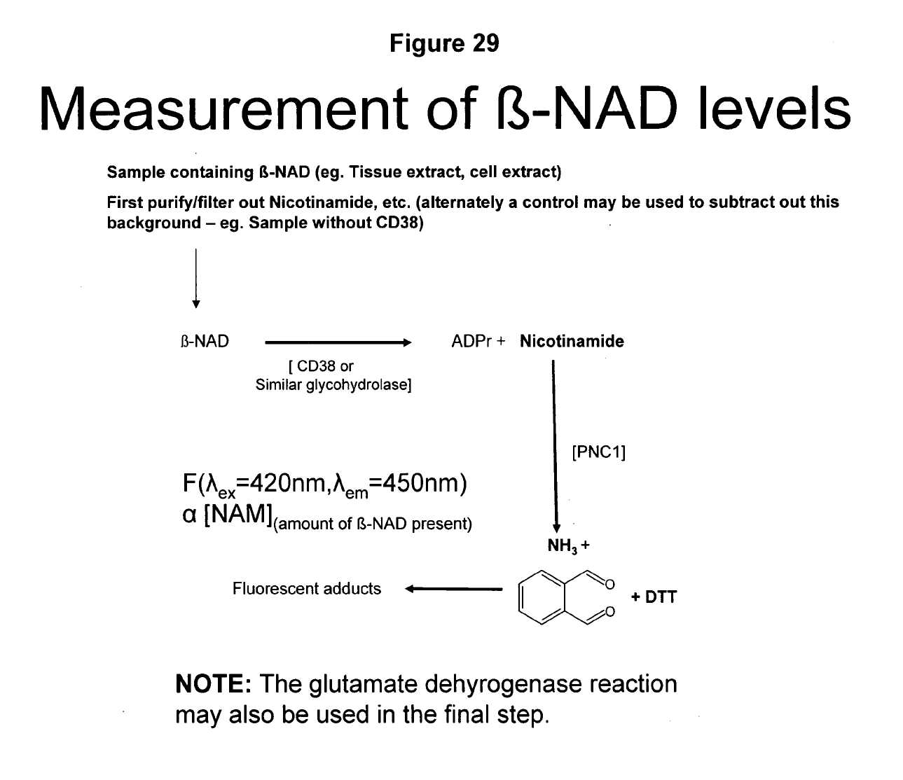

FIG. 30 presents a schematic outlining an assay for measuring the activity of a PBEF/Nampt enzyme.

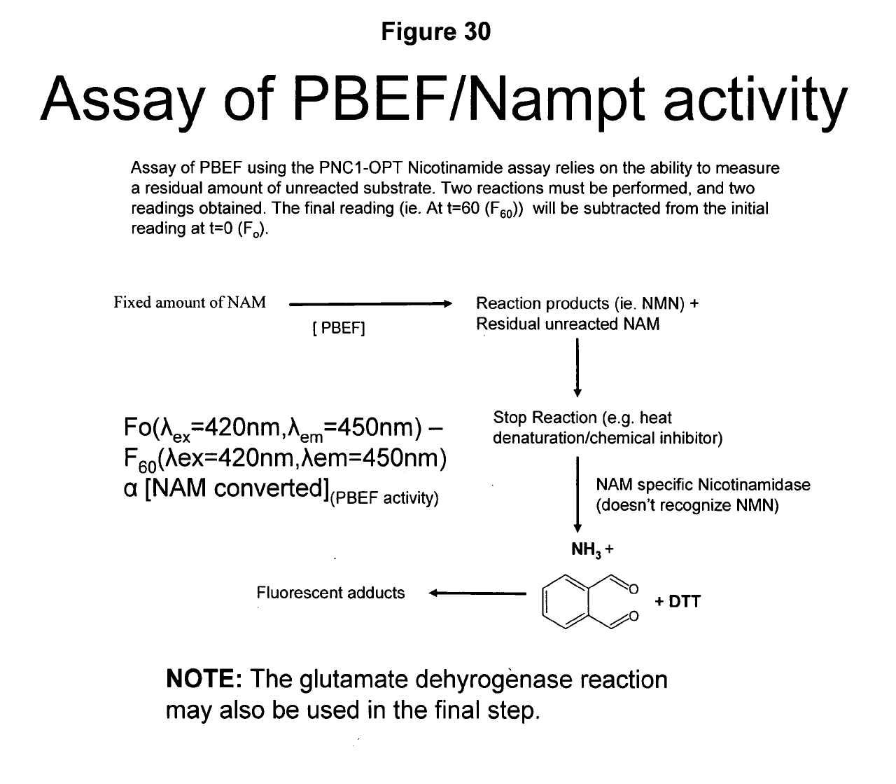

FIG. 31 presents a schematic outlining an assay for measuring the activity of an Nmnat enzyme. The assay can be conducted using Method A in which a NAM specific nicotinamidase which does not recognize NMN is used, or using Method B in which an NMN nicotinamidase is used.

FIG. 32 presents a schematic outlining an assay for measuring the activity of an NRK enzyme. The assay can be conducted using Method A in which a NAM specific nicotinamidase which does not recognize NMN is used, or using Method B in which an NMN nicotinamidase is used.

FIG. 33 presents a schematic outlining an assay for measuring NMN levels. The assay can be conducted using Method A in which a NAM specific nicotinamidase which does not recognize NMN is used, or using Method B in which an NMN nicotinamidase is used.

FIG. 34 presents a schematic outlining an assay for measuring NR levels. The assay can be conducted using Method A in which a NAM specific nicotinamidase which does not recognize NMN is used, or using Method B in which an NMN nicotinamidase is used.

### DETAILED DESCRIPTION

Aspects of the invention relate at least in part to novel methods for measuring nicotinamide producing reactions. Surprisingly, methods described herein offer significant advantages over previous methods for measuring the activity of enzymes that produce nicotinamide. Unlike previous approaches, methods described herein are substrate variable—any substrate may be used in the assays, and no chemical modifications or tags are required. Moreover, the assays are safer (non-radioactive) and more cost effective then other methods currently in use (e.g., mass spectrometry). Methods associated with the invention can be used to measure the activity of various classes of enzymes including deacetylase proteins such as Sirtuins, CD38 and related glycohydrolases, PARPs and mono-ADP-ribosyltransferases, PBEF/Nampt and similar enzymes, nicotinamide mononucleotide adenylyltransferase (NMNAT) and nicotinamide ribose kinases (NRK). Furthermore, methods described herein can be used to identify modulators of enzymes belonging to these classes. Also disclosed are methods for detecting levels of nicotinamide, β-nicotinamide adenine dinucleotide (β-NAD), nicotinamide mononucleotide (NMN) and nicotinamide riboside (NR) in samples. In addition, kits are provided for conducting methods associated with the invention.

This invention is not limited in its application to the details of construction and the arrangement of components set forth in the following description or illustrated in the drawings. The invention is capable of other embodiments and of being practiced or of being carried out in various ways. Also, the phraseology and terminology used herein is for the purpose of description and should not be regarded as limiting. The use of “including,” “comprising,” or “having,” “containing,” “involving,” and variations thereof herein, is meant to encompass the items listed thereafter and equivalents thereof as well as additional items.

Aspects of the invention relate to measuring the activity of enzymes, such as enzymes that produce nicotinamide as a metabolite. In some embodiments, methods relate to measuring the activity of histone deacetylase proteins. Histone deacetylase proteins (HDACs) constitute four different classes. Proteins in class I (Rpd3-like) and class II (Hda1-like) are characterized by their sensitivity to the inhibitor trichostatin A (TSA) (Fischle et al., Biochem Cell Biol 2001, 79(3):337-48; Marks et al., Nature Rev Cancer 2001, 1(3):194-202). Studies using this inhibitor have provided a wealth of information regarding the cellular function of these proteins, including their involvement in the expression of regulators of cell cycle, differentiation, and apoptosis (Yoshida et al. Cancer Chemother Pharmacol 2001, 48(suppl):S20-6. Class III HDACs, which are NADtdependent deacetylases, are known as Sirtuins. Sirtuins are conserved proteins that deacetylate both histone and non-histone cellular targets. In humans, seven sirtuins have been identified (SIRT1-7), with individual Sirtuin proteins exhibiting distinct subcellular localizations and functions.

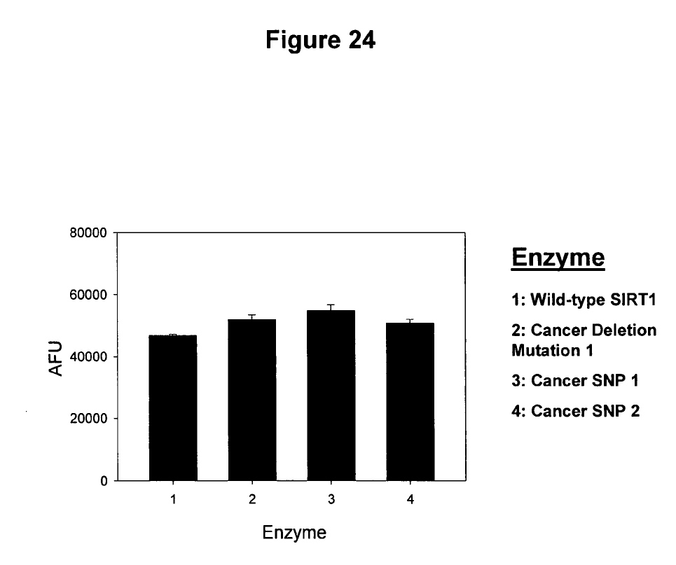

Methods and kits associated with the invention can be applied to measuring the activity of SIRT1, SIRT2, SIRT3, SIRT4, SIRT5, SIRT6 or SIRT7. In some embodiments, methods and kits described herein are used to evaluate the activity of a mutated form of a sirtuin protein. As used herein, a mutated form of a sirtuin protein refers to a sirtuin protein in which the amino acid sequence differs from the wild-type amino acid sequence. For example, a mutated sirtuin protein can have a deletion, an addition, and/or a substitution of one or more amino acids relative to a wild-type protein. In some embodiments, methods and kits associated with the invention are used to assess the change in activity of a mutated sirtuin, such as a sirtuin associated with a disease such as cancer, relative to a wild-type sirtuin protein. FIG. 24 presents analysis of several SIRT1 mutations/deletions found in cancer cell lines.

Yeast Sir2 is the founding member of Class III HDACs. Sir2-like deacetylases are not inhibited by TSA and have the unique characteristic of being NAD⁺-dependent (Smith et al., Proc Natl Acad Sci USA 2000, 97(12):6658-63; Tanner et al., Proc Natl Acad Sci USA 2000, 97(26):14178-82; Landry et al., Proc Natl Acad Sci USA 2000, 97(11):5807-11; Imai et al., Nature 2000, 403(6771):795-800). The biochemistry of Sir2-like deacetylases is reviewed in Moazed, Curr Opin Cell Biol 2001, 13(2):232-8. In vitro, Sir2 has specificity for lysine 16 of histone H4 and lysines 9 and 14 of histone H3 (Smith et al., Proc Natl Acad Sci USA 2000, 97(12):6658-63; Landry et al., Proc Natl Acad Sci USA 2000, 97(11):5807-11; Imai et al., Nature 2000, 403(6771):795-800). Although TSA sensitive HDACs catalyze deacetylation without the need of a cofactor, the Sir2 reaction requires NAD⁺. Sir2 deacetylation is coupled to cleavage of the high-energy glycosidic bond that joins the ADP-ribose moiety of NAD⁺ to nicotinamide. Upon cleavage, Sir2 catalyzes the transfer of an acetyl group to ADP-ribose (Smith et al., Proc Natl Acad Sci USA 2000, 97(12):6658-63; Tanner et al., Proc Natl Acad Sci USA 2000, 97(26):14178-82; Tanny et al., Proc Natl Acad Sci USA 2001, 98(2):415-20; Suave et al., Biochemistry 2001, 40(51):15456-63). The product of this transfer reaction is O-acetyl-ADP-ribose, a metabolite which has been shown to cause a delay/block in the cell cycle and oocyte maturation of embryos (Borra et al., J Biol Chem 2002, 277(15):12632-41). The other product of deacetylation is nicotinamide, a precursor of nicotinic acid and a form of vitamin B3 (Dietrich, Amer J Clin Nut 1971, 24:800-804).

Methods associated with the invention can also be used to measure the activity of NAD⁺ glycohydrolase enzymes. As used herein, an NAD⁺ glycohydrolase enzyme is an enzyme that catalyzes hydrolysis of NAD⁺, leading to production of nicotinamide and ADP-ribose. A non-limiting example of an NAD⁺ glycohydrolase is CD38, an ectoenzyme that is expressed on the surface of immune cells, such as neutrophils; gi:4502665 and GenBank Accession No. NP_{—}001766. In other embodiments, methods associated with the invention can be used to measure the activity of mono or poly (ADP) ribosyltransferases such as poly(adenosine diphosphate-ribose) polymerase-1 (PARP-1), PARPv or tankyrase, enzymes involved in the de novo nicotinamide synthesis pathway.

Aspects of the invention also relate to assays for measuring the activity of the enzyme Nampt, a nicotinamide phosphribosyltransferase enzyme (NAMPRT; E.C.2.4.2.12) that metabolizes nicotinamide. The human gene encoding Nampt is also referred to as pre-B-cell colony enhancing factor 1(PBEF1) and visfatin and exists as two isoforms (Samal et al., Mol Cell Biol 1994, 14:1431; Rongwaux et al., Eur J Immunol 2002, 32:3225; Fukuhara et al., Science 2005, 307:426-30; U.S. Pat. Nos. 5,874,399 and 6,844,163). The sequence of isoform a is available under GenBank Accession numbers NM_{—}005746, NP_{—}005737 and U02020 and the sequence of isoform b is available under GenBank Accession numbers NM_{—}182790, NP_{—}877591 and BC020691. The sequence of a genomic clone of human NAMPRT is provided in GenBank Accession No. AC007032. The structure of the human gene is described in Ognj anovic et al., J Mol Endocrinol 2001, 26:107.

Further aspects of the invention relate to measuring the activity of nicotinamide mononucleotide adenylyltransferase (NMNAT), an enzyme involved in the NAD biosynthetic pathway (Magni et al., Adv Enzymol Relat Areas Mol Biol 1999, 73:135-182. Three different isoforms of NMNAT have been identified in humans: NMNAT 1, 2 and 3, and counterparts have been identified in a variety of species (Emanuelli et al., J Biol Chem 2001, 276:406-412; Schweiger et al., FEBS Lett 2001, 492:95-100; Raffaelli et al., Methods Enzymol 2001, 331; 292-298; Raffaelli et al., Methods Enzymol 2001, 331:281-292; Emanuelli et al., FEBS Lett 1999, 455:13-17; Raffaelli et al., J Bacteriol 1999, 181:5509-5511; Raffaelli et al., Mol Cell Biochem 1999, 193:99-102; Raffaelli et al., Biochem Biophys Res Commun 2002, 297:835-840; Yalowitz et al., Biochem J2004, 377:317-326; Zhang et al., J Biol Chem 2003, 278:13503-13511).

Aspects of the invention also relate to measuring the activity of enzymes that possess nicotinamide ribose kinase activity. As used herein a nicotinamide ribose kinase is a protein which converts nicotinamide riboside to nicotinamide mononucleotide. NRK proteins have been identified in a variety of species including yeast and humans (Bieganowski et al., Cell 2004, 117(4):495-502).

It should be appreciated that methods of the invention permit the use of a wide variety of substrate molecules. In some embodiments, the substrate molecule for the reaction is β-nicotinamide adenine dinucleotide (β-NAD), nicotinamide, nicotinamide mononucleotide (NMN) or nicotinamide riboside (NR). In other embodiments, an additional substrate molecule is added. For example, in embodiments that measure the activity of a deacetylase protein, an acetylated polypeptide is included in the reaction as an additional substrate molecule. A significant advantage of methods described herein is compatibility with a wide variety of substrates. Additionally, substrate molecules do not need to be custom made or modified. Assays described herein can be applied to both peptide substrates and full native substrates. In some embodiments, the substrate is post-translationally modified.

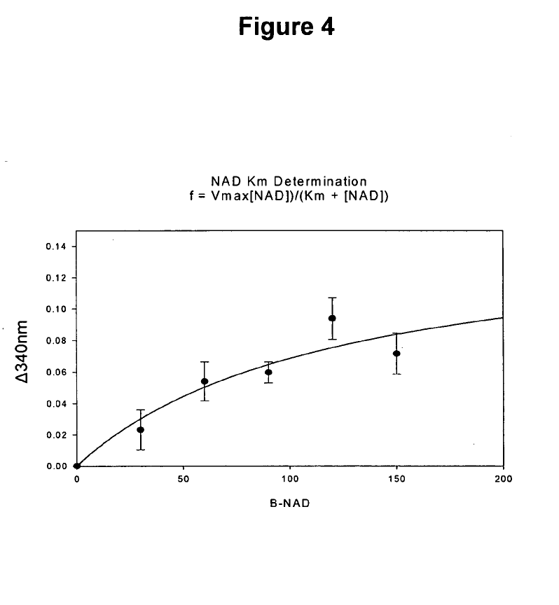

Aspects of the invention relate to screening one or more substrates to identify substrate-specific activators and/or substrate-specific inhibitors. In some embodiments, a substrate is tagged, while in other embodiments, a substrate is not tagged. In certain embodiments, a substrate is a whole protein (FIG. 4). One or more substrates can be screened individually or in groups such as in a library of substrates.

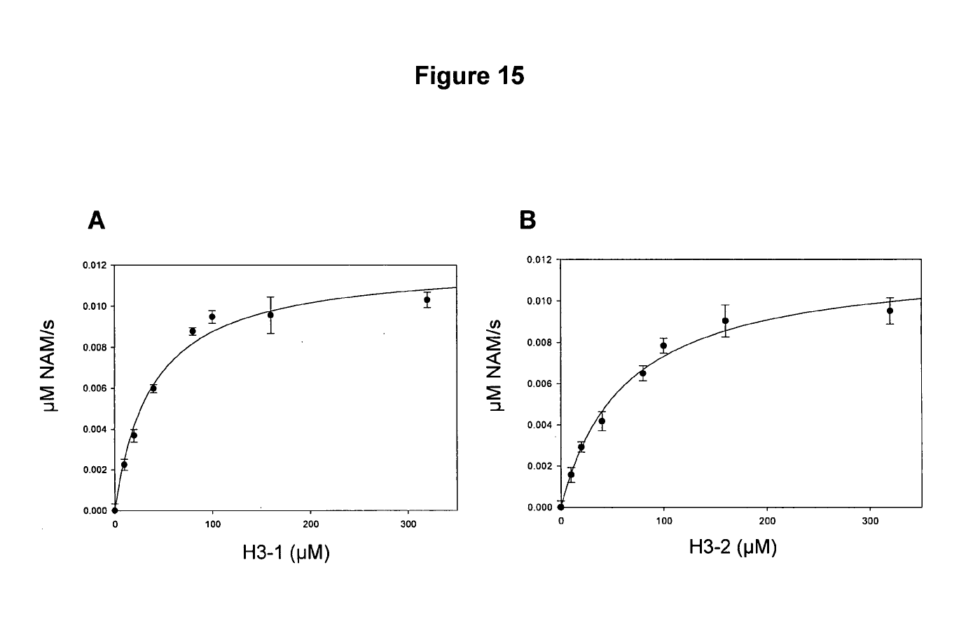

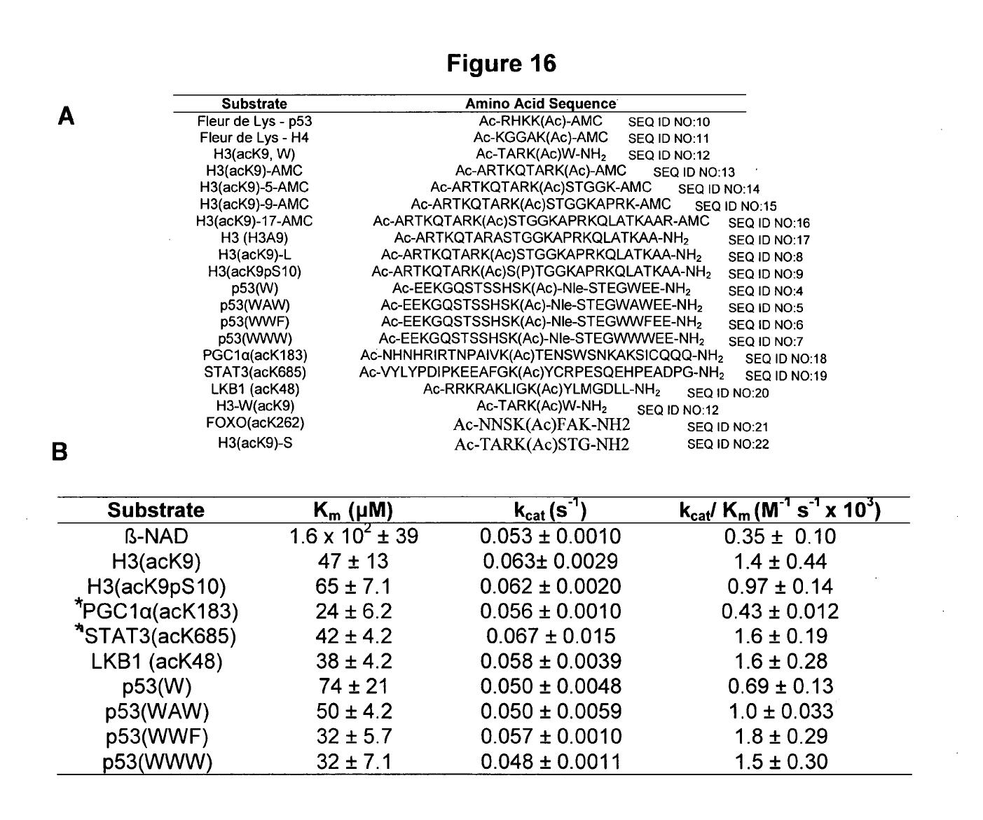

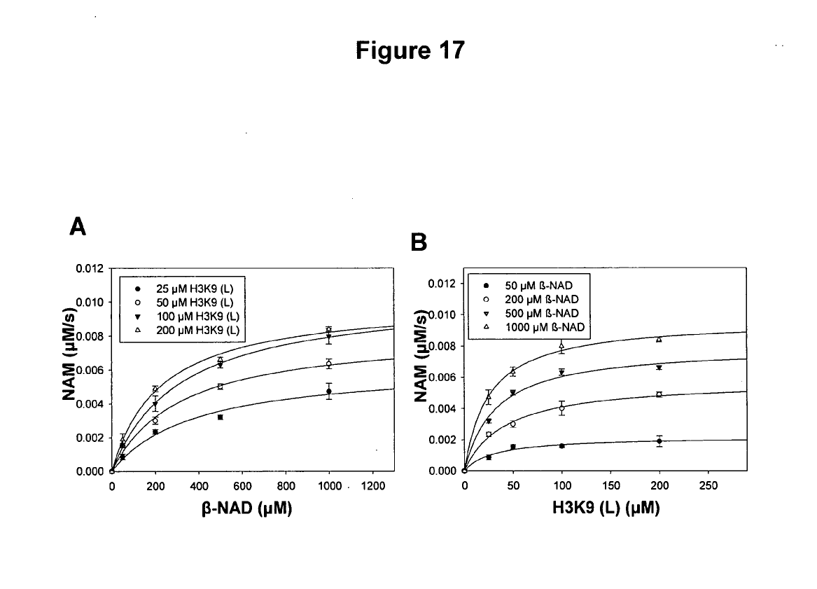

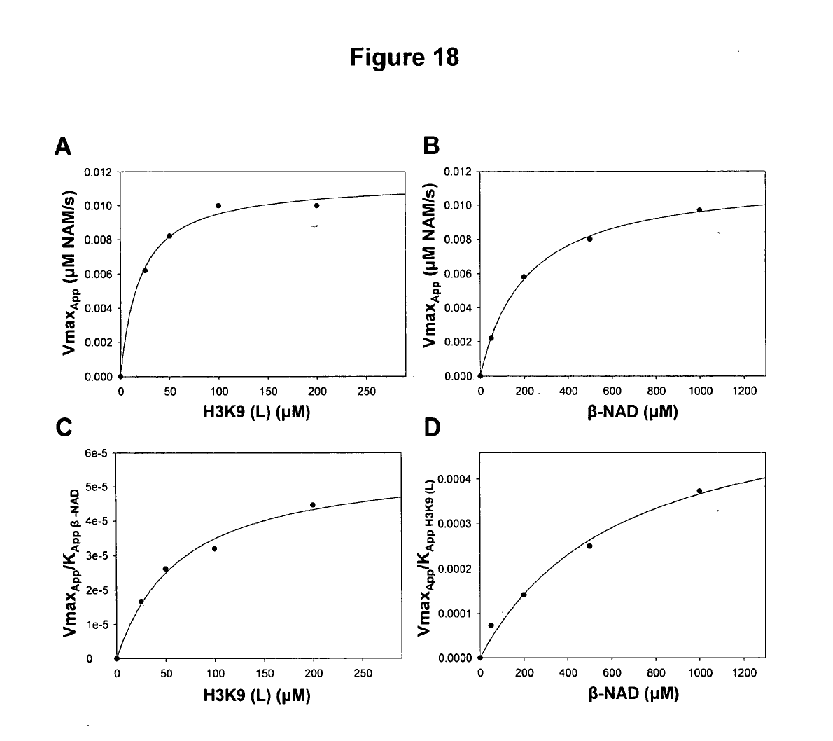

Further aspects of the invention relate to determining substrate preference for an enzyme such as a sirtuin and determining kinetic parameters of an enzyme for a substrate, such as a sirtuin substrate. FIG. 15 demonstrates Km determination for two substrate peptides. FIG. 16 presents parameters for a range of different substrate molecules. Sirtuin deacetylase activity can be measured under varying substrate and β-NAD concentrations and Michaelis-Menten curves can be generated for β-NAD at various fixed concentrations of the substrate, and for the substrate at various fixed concentrations of β-NAD (FIG. 17). Secondary analysis plots of Vmax and Vmx/K vs substrate and β-NAD can then be generated (FIG. 18).

It should be appreciated that such analysis can be used to perform mechanistic studies of enzyme-substrate interactions. For example, data such as that presented in FIGS. 17 and 18 can be used to investigate whether the activity of an enzyme on a substrate is sequential (random or ordered) or ping-pong, as would be understood by one of ordinary skill in the art.

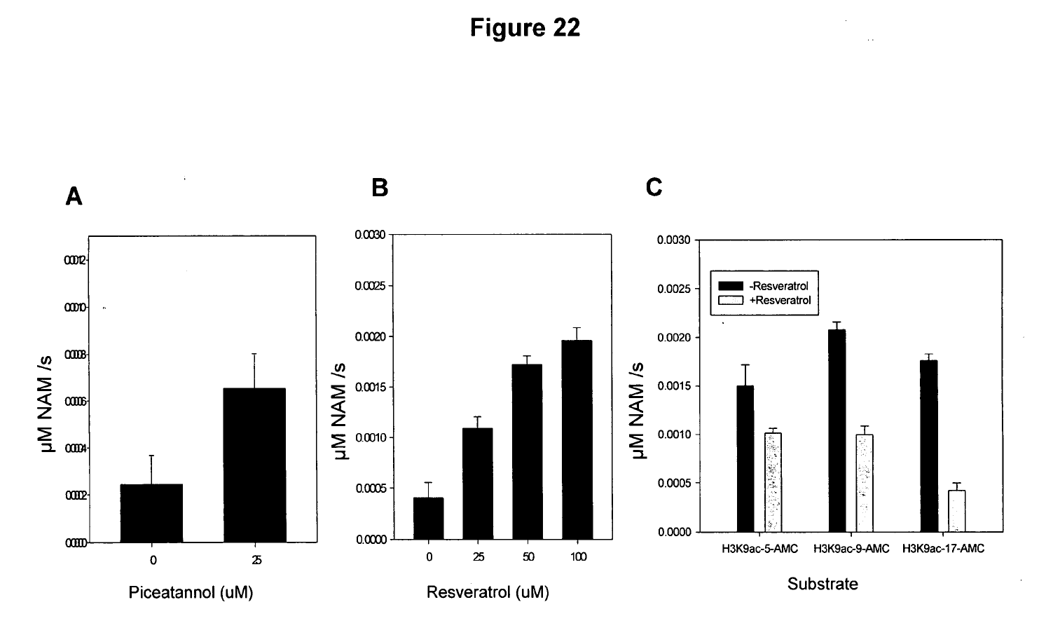

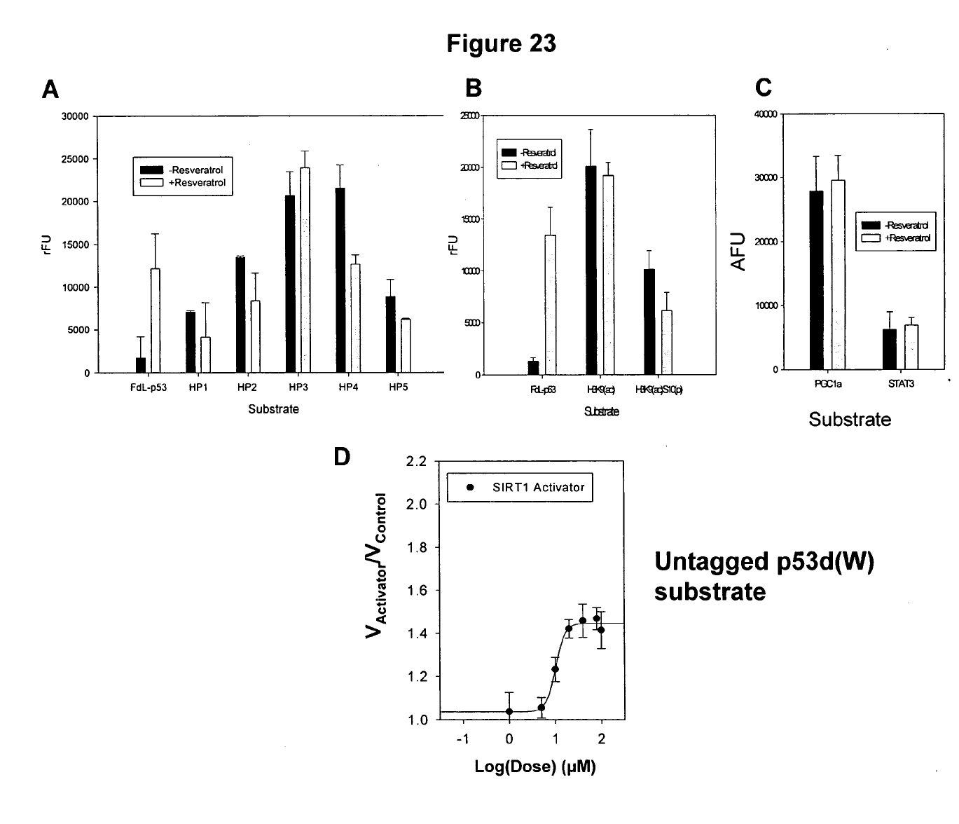

An analysis of AMC-tagged substrates is presented in FIG. 22. On an AMC-tagged substrate, the AMC moiety can be located at various distances from the acetylated lysine residue. For example, in some embodiments, the AMC moiety is located 1, 2, 3, 4, 5, 6, 7, 8, 9, 10, 11, 12, 13, 14, 15, 16, 17 or more than 17 amino acids away from the acetylated lysine residue. FIG. 23 presents data involving native (non-tagged) and tagged peptide substrates. FIG. 23D demonstrates activation of a non-fluorophore tagged substrate using a SIRT1 activator.

Aspects of the invention relate to the use of nicotinamidase proteins for converting nicotinamide to ammonia. In some embodiments, the nicotinamidase protein is a PNC1 protein or a homolog thereof A nucleotide sequence encoding S. cerevisiae PNC1 and the protein encoded thereby are represented by GenBank Accession numbers NC_{—}001139 and NP_{—}011478, respectively. PNC1 is the yeast homologue of the bacterial protein pncA, which catalyzes the hydrolysis of nicotinamide to nicotinic acid and ammonia. S. cerevisiae PNC1, also referred to as open reading frame (ORF) YGL037 is described in Ghislain et al., Yeast 2002, 19:215. The nucleotide and amino acid sequences of an Arachis hypogaea PNC1 is provided by GenBank Accession numbers M37636 and AAB06183 and are described in Buffard et al., Proc Natl Acad Sci USA 1990, 87:8874. Nucleotide and amino acid sequences of related human proteins are provided by GenBank Accession numbers BC017344 and AAH17344, respectively; AK027122 and NP_{—}078986, respectively; XM_{—}041059 and XP_{—}041059, respectively; and NM_{—}016048 and NP_{—}057132, respectively. A Drosophila PNC1 homolog is represented by AAF55694.

A human functional homolog of PNC1 is NAMPRT, also called Nampt/PBEF/visfatin, described above. Unlike yeast PNC1, human PBEF protein converts nicotinamide into nicotinamide mononucleotide, rather than ammonia. In some embodiments of the invention, the nicotinamidase catalyzed step of the method can be achieved by a multi-enzyme mixture, for example a mixture consisting of multiple mammalian proteins that together convert nicotinamide into ammonia.

It should be appreciated that all nicotinamidase enzymes that convert nicotinamide into ammonia, are consistent with methods described herein. For example, any protein that is found to be functionally equivalent to PNC1 would be compatible with methods of the invention. A functional equivalent of PNC1, as used herein is a protein from any species, or a synthetic protein, that is capable of converting nicotinamide to ammonia in a manner comparable to that of PNC1. Assays for determining the activity of a PNC1 protein are described, e.g., in Ghislain et al., Yeast 2002, 19:215-224. In this reference, the authors identified yeast PNC1 through its ability to bind to a Ni²⁺ agarose bound resin. They confirmed the activity of the enzyme using two methods: 1) a GDH-based kit available from Sigma-Aldrich, St. Louis, Mo.) and 2) using a pyrazinamide (PZA) assay. The PZA assay is described in Frothingham et al., Antimicrobial Agents and Chemotherapy 1996, 40:1426-1431. In addition to nicotinamide, PNC1 proteins have activity on other substrates such as nicotinyl hydroxamic acid and PZA, an antibiotic used in the treatment of tuberculosis. Methods involving any substrate of PNC1 proteins are compatible with aspects of the invention.

One of ordinary skill in the art would be able to determine, based for example on in vitro, in vivo, or cell-based assays, whether a given protein is a functional equivalent of PNC1. Several non-limiting examples of approaches for identifying functional equivalent of PNC1 include screening for enzymes which fortuitously bind to Ni²⁺ agarose bound resin as in Ghislain et al., Yeast 2002, 19:215-224; screening for genes which disrupt PZA processing as in Frothingham et al., Antimicrobial Agents and Chemotherapy 1996, 40:1426-1431; screening for genes which disrupt nicotinamide metabolism in general, or β-NAD meatabolism (e.g., changes in the levels of metabolites, or their downstream effectors). Enzymatic activity of putative functional equivalents of PNC1 could be subsequently tested by assays such as those use in Ghislain et al., Yeast 2002, 19:215-224.

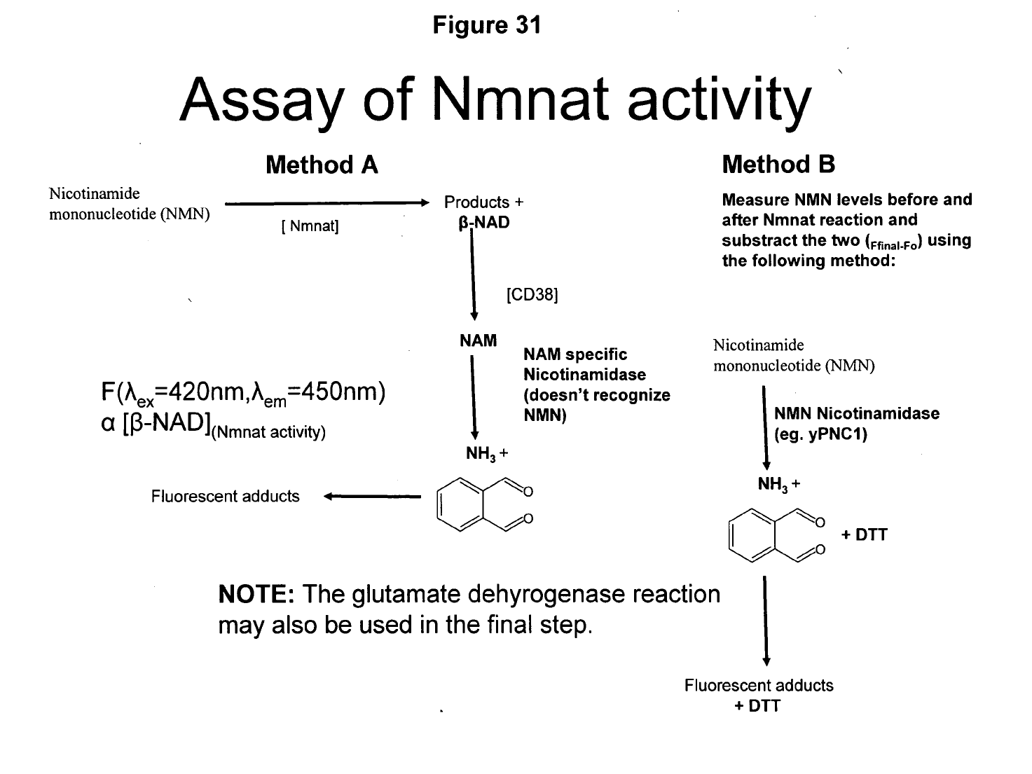

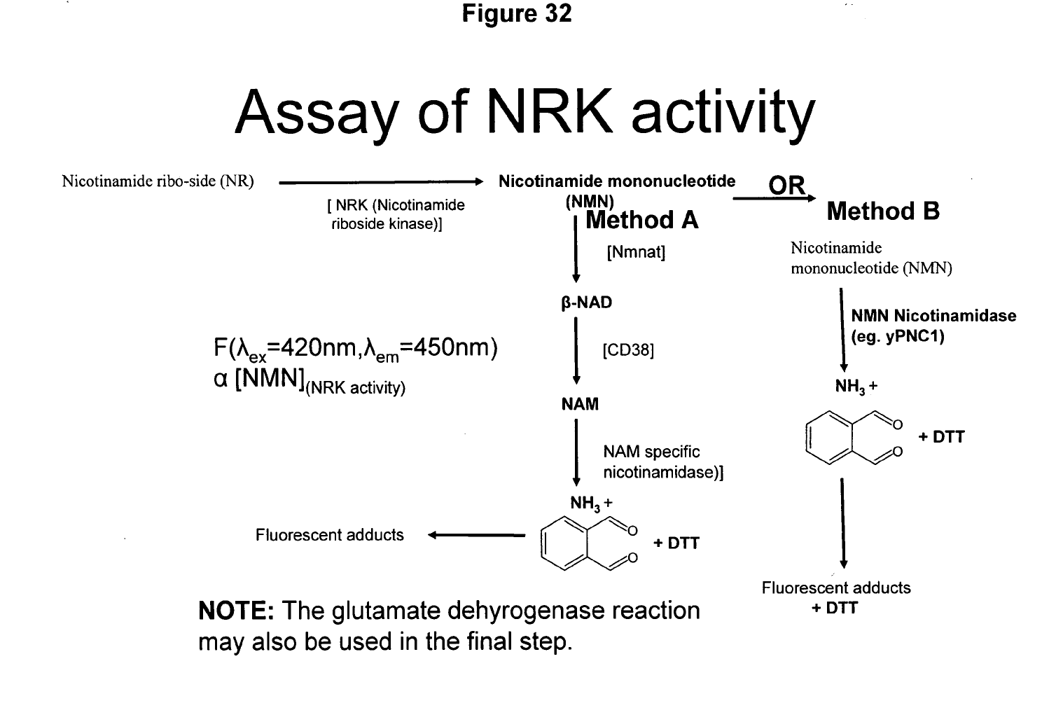

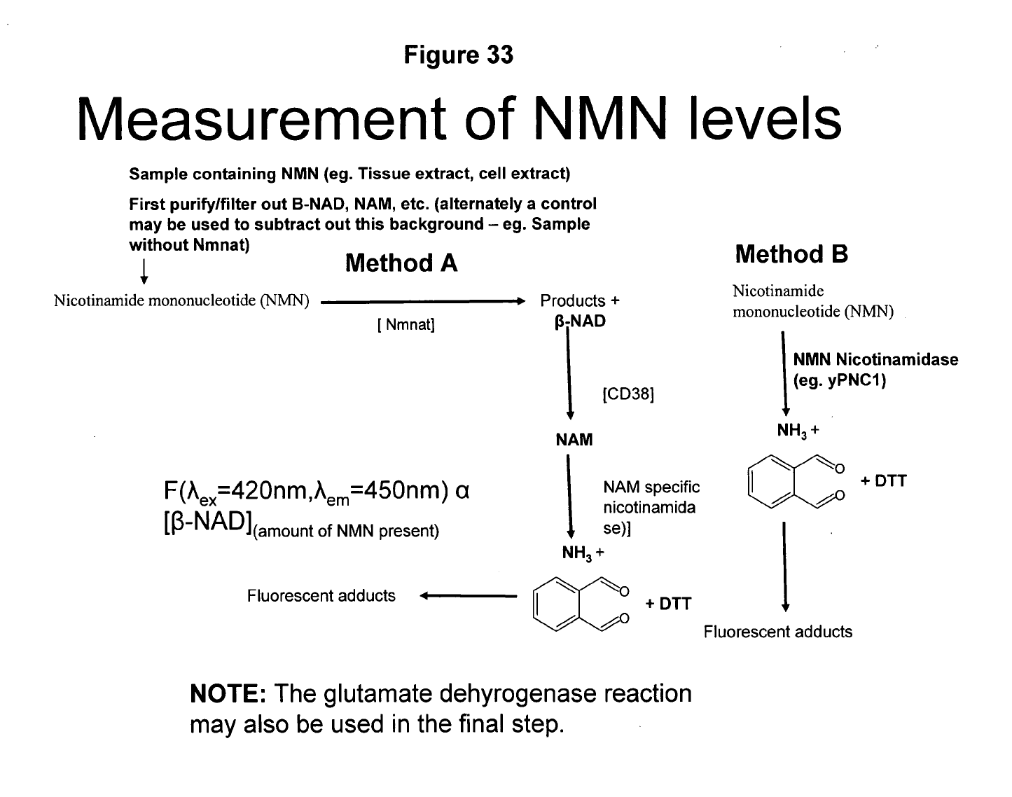

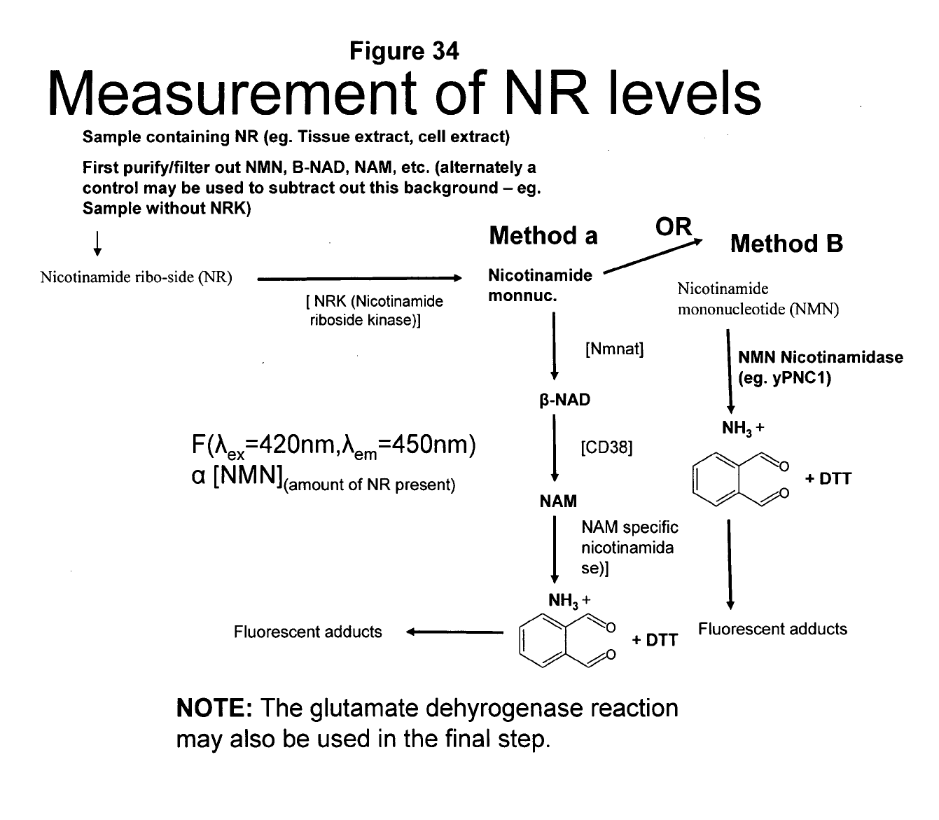

It should be appreciated that in some embodiments, the nicotinamidase protein, such as a PNC1 protein, is specific for NAM and does not recognize Nicotinamide mononucleotide (NMN). In other embodiments, the nicotinamidase protein, such as a PNC1 protein, does recognize NMN. One of ordinary skill in the art would understand how to determine, using assays described herein and knowledge in the art, whether a nicotinamidase protein recognizes NMN or not. It should also be appreciated that nicotinamidase proteins that recognize NMN and those that do not recognize NMN are both compatible with methods described herein as demonstrated in FIGS. 31-34.

The yeast nicotinamidise PNC1 has been shown to convert nicotinamide into ammonia in vivo in yeast, and to relieve Sirtuin inhibition (Anderson et al., Nature 2003, 423(6936):181-5). It has also been shown in vivo that nicotinamide acts as an endogenous inhibitor of Sir2, and that yPNC1 is able to activate Sir2 by mitigating this effect. (Id.) By exploiting the ability of this enzyme (or other NAD-cleaving enzyme) to convert the downstream product of a Sirtuin reaction, nicotinamide, into ammonia, assays described herein allow measurement of the rate of an enzymatic reaction, such as a Sirtuin deacetylation reaction, based on the amount of ammonia produced following the addition of a nicotinamidase enzyme such as yPNC1 in excess, i.e., in an amount sufficient to convert all nicotinamide in the sample to which the enzyme is added to ammonia within a selected time.

In some embodiments, the reaction that produces nicotinamide is terminated prior to addition of a nicotinamidase enzyme such as PNC1. For example, if the reaction that produces nicotinamide is a deacetylation reaction, the reaction may be terminated by removal of the deacetylase enzyme such as by filtration (or column centrifugation), or chemical or heat inhibition of the enzyme, or any other means known to one of ordinary skill in the art. In other embodiments, the reaction that produces nicotinamide is not terminated prior to addition of the nicotinamidase enzyme such as PNC1. For example, in some embodiments, the enzyme is inactivated and/or removed for end-point measurements, while in other embodiments, the assay is run in a continuous mode without inactivation of the enzyme. In such cases, absorbance measurements may be taken continuously. In some embodiments, the nicotinamidase enzyme such as PNC1 is added simultaneously with the components of the reaction that produces nicotinamide. Addition of the nicotinamidase enzyme such as PNC1 converts essentially all of the nicotinamide into ammonia.

Any means of detecting ammonia is compatible with methods described herein. In some embodiments, the amount of ammonia is assayed using a glutamate dehydrogenase (GDH) reaction (Cheuk et al., J Agric Food Chem 1984, 32:14-18; Mondzac et al., J Lab & Clin Med 1965 66:526-531; Van Anken et al., Clinical Chemica Acts 1974, 56:151-157; Neeley et al., Clin Chem 1988, 34:1868-1869). A coupling of the PNC1 reaction to the GDH reaction, as described in Example 1, allows for the robust one-step detection of NAM levels. Previously it has been shown that glutamate dehydrogenase has the ability to catalyze the conversion of a-ketoglutarate and NADPH (nicotinamide adenine dinucleotide phosphate reduced) into L-glutamate and NADP (oxidized), in the presence of ammonia. (Id.) This enzymatic pathway has been applied successfully to the in vitro measurement of ammonia levels in various food products, and biological fluids. (Id.)

In some embodiments, the amount of ammonia is assayed using a fluorescent assay, an example of which is described in Example 2. Ammonia can be detected, for example, via reaction with o-phthalaldehyde as described in Sugawara et al., J Biochem 1981, 89:771-774; Corbin, Applied Environmental Microbiology 1984, 1027-1030. In some embodiments, the products of the PNC1 reaction are reacted with o-phthaladehyde in the presence of a reducing agent. Several non-limiting examples of reducing agents include DTT, β-mercaptoethanol, thioglycolic acid, and sodium hydrosulfite. Reaction of ammonia with o-phthaladehyde in the presence of a reducing agent leads to the production of fluorescent adducts. The fluorescent adducts obtained are measured fluorometrically with excitation at approximately 413 nm and emission at approximately 476 nm. This reaction has been reported to be specific for ammonia (Sugawara et al., J Biochem 1981, 89:771-774). A blank reaction is also performed in the absence of NAD.

It should be appreciated that molecules other than o-phthaladehyde can be used, according to aspects of the invention, to react with ammonia and produce quantifiable fluorescent products. Any other molecule that reacts with amines, and that produces a discrete set of wavelengths when it reacts with ammonia, allowing quantification of the specific fluorescent product(s) produced, would be compatible with methods described herein.

It should also be appreciated that any other means of detecting ammonia is also compatible with methods described herein. Methods for detecting ammonia or ammonium are described in, and incorporated by reference from, U.S. Pat. No. 5,076,904 and U.S. Pat. No. 5,014,009, including gas chromatography for ammonia/amine compounds. Other means for detecting ammonia include the Ninhydrin reaction, described in, and incorporated by reference from, Harding et al., J Biol Chem 1916, 25:319-335 and the Indophenol Blue Reaction, available from Turner Designs, Sunnyvale, Calif. (S-0025; Ammonium Concentration Determination).

Further aspects of the invention relate to using methods described herein to screen for modulators such as activators or inhibitors of enzymes described herein. In some embodiments, activators or inhibitors of enzymes described herein are small molecule activators or inhibitors. As one of ordinary skill in the art would appreciate, screening can be conducted on a small scale or in a high-throughput format. It should be appreciated that methods described herein can be carried out in any suitable reaction size and any suitable reaction vessel or container. In some embodiments, the assay is performed in individual cuvettes, such as 1 mL cuvettes. In some embodiments, the assay is conducted in a multi-well plate such as a 96-well plate. In some embodiments, the effects of post-translational modifications or protein binding partners on the enzymatic activity of enzymes described herein or modulators of enzymes described herein, can also be investigated using methods associated with the invention.

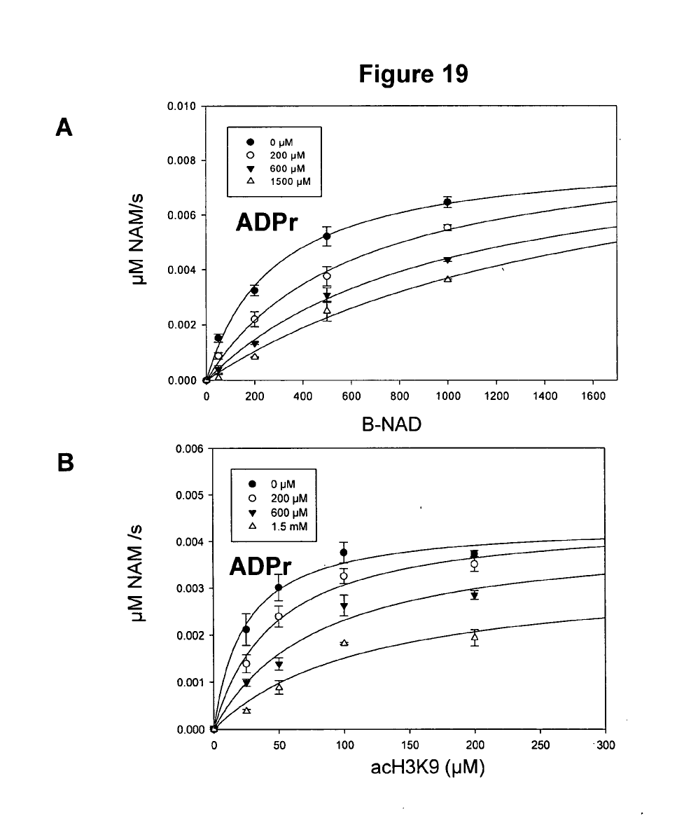

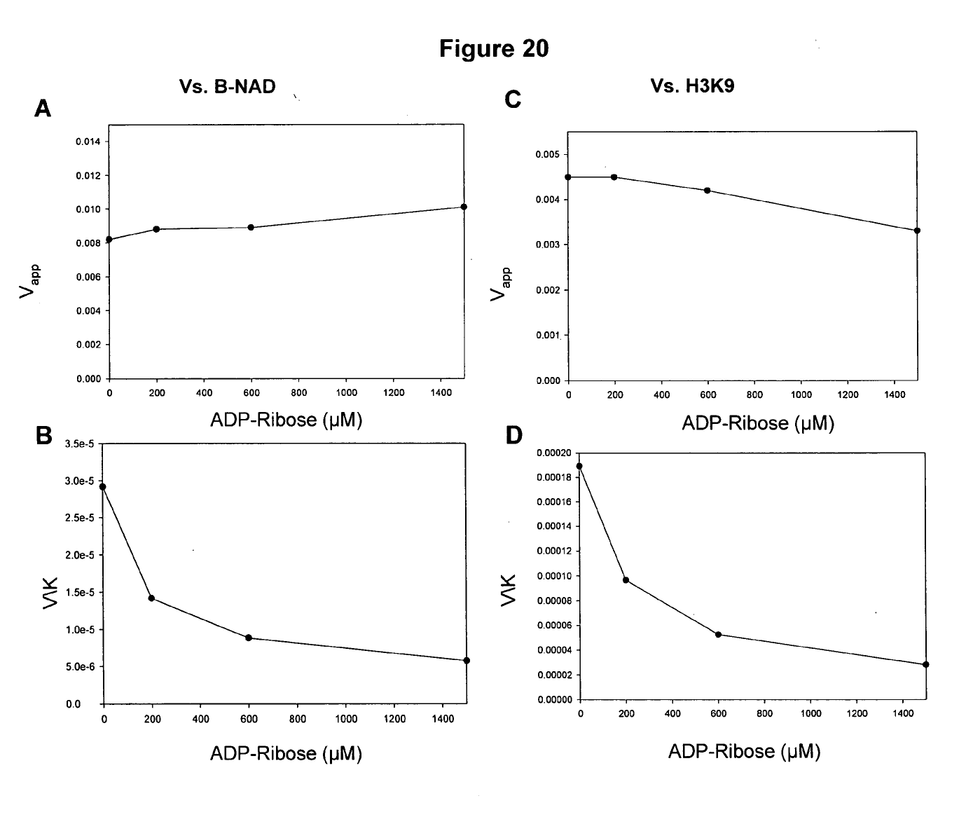

Methods described herein can also be used to perform mechanistic studies of activators and/or inhibitors of enzymes such as sirtuins. Michaelis-Menten curves can be generated for β-NAD, and for a substrate of the reaction, at various concentrations of an activator or an inhibitor (FIG. 19). Secondary analysis plots of Vmax and Vmax/K vs the concentration of the inhibitor or activator for both β-NAD and the substrate can be generated (FIG. 20). Results of such analysis can be used, for example, to determine whether an inhibitor, such as a sirtuin inhibitor, acts as a competitive or non-competitive inhibitor.

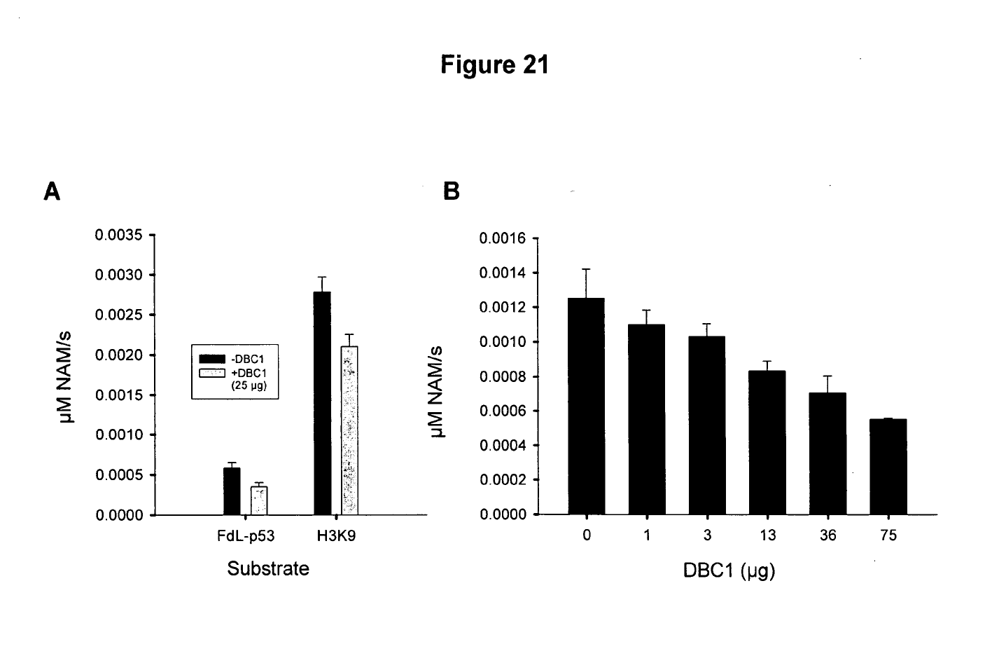

In some embodiments, an activator or an inhibitor of an enzyme such as a sirtuin is an endogenous protein. For example, DBC1 and AROS are known inhibitors and activators respectively of SIRT1 (FIG. 21). Any of the methods described herein for characterizing activators and/or inhibitors of enzymes, can be applied to activators and/or inhibitors that are endogenous proteins.

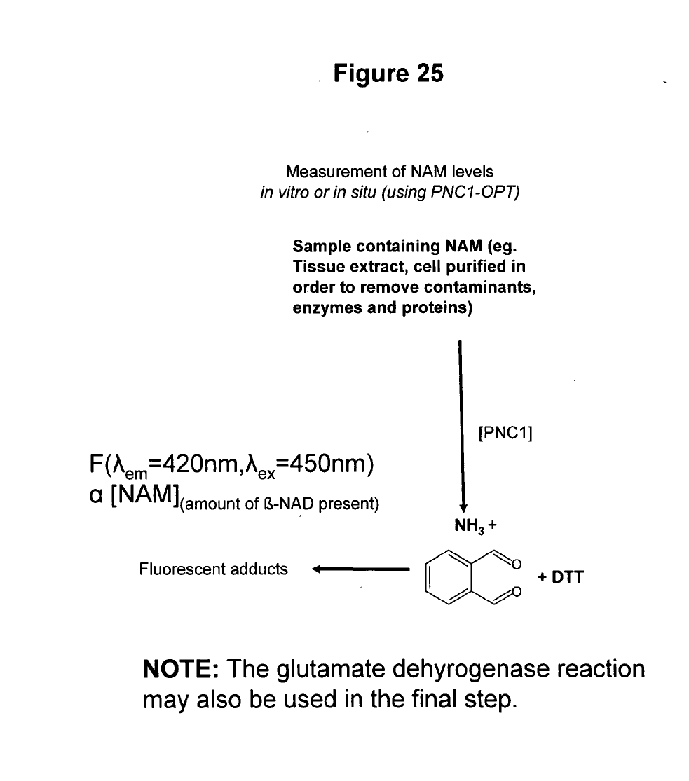

Further aspects of the invention relate to using methods described herein to measure the amount of a molecule in a sample. Several non-limiting examples of molecules that can be measured using methods described herein include nicotinamide, β-nicotinamide adenine dinucleotide (β-NAD), nicotinamide mononucleotide (NMN) and nicotinamide riboside (NR) in samples. It should be appreciated that according to methods described herein, such molecules can be measured in a wide variety of samples including but not limited to water samples, food samples, tissue samples, cell samples or soil samples. For example, an assay for measuring nicotinamide levels in vitro or in situ is presented in FIG. 25.

It should be appreciated that according to aspects of the invention, measured values are compared to control values where appropriate. In some embodiments, a control is a predetermined value, which can take a variety of forms. It can be a single cut-off value, such as a median or mean or it can be established based upon comparative groups. Appropriate controls, including in some embodiments, predetermined ranges and categories can be selected with no more than routine experimentation by those of ordinary skill in the art. In some embodiments, controls according to aspects of the invention may be, in addition to predetermined values, samples of materials tested in parallel with the experimental materials. Examples include in some embodiments, blank reactions carried out in the absence of NAD, in the absence of (a polypeptide) substrate, in the absence of a test molecule or in the absence of an enzyme tested in parallel with the experimental samples. In some embodiments, control values are subtracted from test values in order to normalize data. Positive controls, such as parallel reactions containing reaction components and a known amount of active enzyme also can be run in order to confirm assay function.

The invention also contemplates the use of kits, such as kits for measuring the activity of a given enzyme. An example of such a kit may include a nicotinamidase protein such as PNC1 and a molecule such as o-phthalaldehyde for reaction with ammonia. In some embodiments the kit may further comprise one or more enzymes such as a deacetylase, CD38 or a related glycohydrolases, a PARP or mono-ADP-ribosyltransferases, PBEF/Nampt or similar enzyme, nicotinamide mononucleotide adenylyltransferase (NMNAT) or nicotinamide ribose kinases (NRK). In some embodiments, a kit may comprise nicotinamide, β-NAD, nicotinamide mononucleotide (NMN), nicotinamide riboside (NR), and/or an additional substrate molecule. In some embodiments, a kit may comprise one or more of a-Ketoglutarate, NADPH, and glutamate dehydrogenase.

In some embodiments, the kit is a kit for measuring activity of a sirtuin. A representative kit for measuring sirtuin activity was developed and tested as described in Example 4. In some embodiments, a kit for measuring the activity of a sirtuin contains one or more of the following components: an assay buffer, a peptide substrate (tagged or untagged), β-NAD, a sirtuin enzyme such as hSIRT1, a nicotinamidase enzyme such as γPNC1, a developing reagent, nicotinamide and one or more sirtuin inhibitors or activators. Experimental procedures for use with such a kit are further described in Example 4.

Kits for screening for modulators of enzymes described herein are also compatible with aspects of the invention, as are kits for identifying the presence of molecules described herein in a given sample. Kits associated with the invention may further comprise instructions for use of components of the kit. In some embodiments, kits may comprise one or more containers containing the foregoing reaction components and may also comprise further reagents or buffers including solvents, surfactants, preservatives and/or diluents.

The compositions of the kit may be provided as any suitable form, for example, as liquid solutions or as dried powders. When the composition provided is a dry powder, the composition may be reconstituted by the addition of a suitable solvent, which may also be provided. In embodiments where liquid forms of the composition are used, the liquid form may be concentrated or ready to use. The solvent will depend on the composition and the mode of use or administration. Suitable solvents for such compositions are well known, and are available in the literature. The solvent will depend on the composition and the mode of use.

The present invention is further illustrated by the following Examples, which in no way should be construed as further limiting. The entire contents of all of the references (including literature references, issued patents, published patent applications, and co-pending patent applications) cited throughout this application are hereby expressly incorporated herein by reference.

### EXAMPLES

### Example 1

### Non-Fluorometric Nicotinamide Assay

A SIRT1 enzymatic deacetylation reaction was carried out in the presence of an acetylated substrate and β-NAD. Following an arbitrary time interval at 37° C., the deacetylation reaction was terminated either by removal of SIRT1 by filtration (or column centrifugation), or chemical or heat inhibition of the enzyme. Subsequently, a-Ketoglutarate and NADPH were added to the reaction mix. The absorbance was read at 340 nm using an appropriate spectrophotometer (Rₒ). PNC1 and GDH were then added in excess to induce the coupled conversion of NADPH to NADP (PNC1 first converts NAM to NH3, and subsequently GDH uses the ammonia to oxidize NADPH to NADP). A second reading at 340 nm was then obtained (R₁). The difference in absorbance readings, R₁-Rₒ is proportional to the amount of NAM produced in the initial deacetylation reaction, thereby allowing for the measurement of activity. A blank reaction was carried out in the absence of NAD (or absence of Enzyme), as above, and subtracted from each sample in order to normalize the data.

### Methods

Reactions, adapted for use with SIRT1, were carried out as described in the following protocol:

### SIRT1 Nicotinamide Assay Protocol

Ex. Substrate Concentration (BIOMOL)=20 uM; NAD Concentration=1 mM

### Reaction 1 (SIRT1 Deacetylation Reaction): Acetyl-peptide→Nicotinamide

1) Mix the following (final volume 100 uL):
a) 10 uL 10×BIOMOL Buffer (250 mM Tris-Cl [pH=8], 1.37 M NaCl, 27 mM KCl, 10 mM MgCl₂)
b) 0.4 uL *H4K16-Lys BIOMOL peptide (from a 5 mM stock for a final concentration of 20 uM)
c) 1 uL of 100 mM B-NAD (for a final concentration of 1 mM)
d) 2 uL SIRT1 enzyme (at 1.2 ug/ul, total ˜2.5 ug)
e) 83 uL ddH20
   2) Mix the reaction by pipetting. Incubate at 37° C. for the appropriate amount of time.
   3) Stop the reaction by heating at 95° C. for 5 minutes. Place the reaction on ice until ready to proceed with reaction 2 (Centrifuge Briefly).

### Reaction 2: Nicotinamide→Ammonia→Oxidation of NADPH

4) For each reaction make 85 uL of Conversion reagent as follows (fresh each time):
a) 20 uL a-Ketoglutarate (100 mM Stock in dPBS) (for a final concentration of 10 mM)
b) 0.7 uL NADPH (100 mM Stock in dPBS) (for a final concentration of 350 uM in 200 uL)
c) 5 uL DTT (Stock is 45 mM) (for a final concentration ˜1 mM)
d) 59 uL dPBS
   5) Read the absorbance at 340 nm (Ao)

### 6) Sequentially add:

a) 5 uL PNC1 (3 ug of enzyme at 0.6 ug/uL)
b) 10 uL Sigma GDH (G2294)
   7) Mix well by pipetting up and down 3 times (make sure no bubbles are present). Incubate at room temperature (18-30° C.) for 15 minutes.
   8) Read the absorbance at 340 nm (Ai)
   9) Subtract Ai-Ao, and use standard curve to calculate the amount of NAM produced.

### REFERENCES

1. Cheuk, W. L. and Finne, G. Enzymatic Determination of Urea and Ammonia in Refrigerated Seafood Products. J. Agric. Food Chem., 32, 14-18 (1984).
2. Mondzac, A., et al., An Enzymatic Determination of Ammonia in Biological Fluids. J. Lab. & Clin Med., 66, 526-531 (1965).
3. Van Anken, H. C. and Schiphorst, M. E., A. Kinetic Determination of Ammonia in Plasma, Clinical Chemica Acts, 56, 151-157 (1974).
4. Neeley, W. E., and Phillipson, J. Automated Enzymatic Method for Determining Ammonia in Plasma, with 14-day Reagent Stability. Clin. Chem., 34, 1868-1869 (1988).
5. Anderson et al. Nicotinamide and PNC1 govern lifespan extension by calorie restriction in Saccharomyces cerevisiae. Nature, 423(6936):181-5 (2003)
6. Howtiz et al., Small molecule activators of sirtuins extend Saccharomyces cerevisiae lifespan. Nature 425(6954):191-6 (2003).
7. Borra et al., Mechanism of human SIRT1 activation by resveratrol. Journal of Biological Chemistry 280(17):17187-95 (2005).
8. Kaeberlein et al. Substrate-specific activation of sirtuins by resveratrol. Journal of Biological Chemistry 280(17):17038-45 (2005).
9. Milne et al., Small molecule activators of SIRT1 as therapeutics for the treatment of type 2 diabetes. Nature 450(7170):712-6 (2007).

### Example 2

### Fluormetric PNC1/OPT-Based Nicotinamide Assay

A SIRT1 enzymatic deacetylation reaction was carried out in the presence of an acetylated substrate and β-NAD. Following an arbitrary time interval at 37° C., the deacetylation reaction was terminated either by removal of SIRT1 by filtration (or column centrifugation), or chemical or heat inhibition of the enzyme. Subsequently, PNC1 was added to the mixture to convert all of the nicotinamide generated during the reaction into ammonia. Additionally, PNC1 may be added directly to the SIRT1 reaction (and the reactions may proceed simultaneously). Ammonia was detected via reaction with o-phthalaldehyde as described in Sugawara and Oyama (1981) and Corbin (1984). In brief, the products of the PNC1 reaction were reacted with o-phthaladehyde in the presence of DTT. The reaction was allowed to proceed for 1 hour, and subsequently the amount of fluorescent adducts obtained were measured fluorometrically with excitation at ˜413 nm and emission at ˜476 nm. This reaction has been reported to be specific for ammonia (Sugawara and Oyama, 1981). A blank reaction was also performed in the absence of NAD.

### Methods

Reactions, adapted for use with SIRT1, were carried out as described in the following protocol:

### SIRT1 PNC1-OPT Nicotinamide Assay Protocol

### Reaction 1 (SIRT1 Deacetylation Reaction): Acetyl-peptide→Nicotinamide

Prepare a master-mix for the total number of reactions you wish to perform. For each individual reaction, mix the following in the order presented (the total volume for each reaction is 100 uL):

a) 10 uL 10×BIOMOL SIRT1 Assay Buffer (250 mM Tris-Cl [pH=8], 1.37 M NaCl, 27 mM KCl, 10 mM MgCl₂)
b) distilled/deionized H₂0 (100 uL—volume of the other components)
c) Substrate (typical peptide concentrations vary between 10-100 uM; native proteins may be used)
d) β-NAD (typical β-NAD levels vary between 10-200 uM)
e) Purified yeast PNC1 enzyme (˜2 ug)
f) Purified Sirtuin enzyme (˜2 ug)
2) Mix the reaction by pipetting, and subsequent gentle vortexing (setting 6 or 7). Incubate at 37° C. for the appropriate amount of time (typically 1 hour)
Reaction 2: Nicotinamide→Ammonia→Fluorescent adducts
Note: When adding the OPT Developer it is advisable to work in a dark or dimly lit setting. Dim the lights prior to adding the developer to each reaction.
a) Add 100 uL of the OPT Developer Mix to each reaction.
b) Mix each sample by vortexing briefly for 5 s at the highest setting.
c) Immediately cover all of the samples with Aluminum foil, and incubate the reactions on an orbital shaker for 1 hour at room temperature. Once covered, ambient lighting may be restored.
3) If necessary samples may be transferred to a 96-well plate for reading (do this under dim light). Read the fluorescence on a fluorometer with filters set to excitation (˜420 nm+/−20, and emission ˜450 nm (+/−20). A 0.1 s or 1 s read is generally recommended.

### Solutions

OPT Developer Reagent: A solution of 10 mM OPT and 10 mM DTT in 100% EtOH. Store at −20° C. in the dark until use. Cover with Aluminum foil in order to minimize exposure to light.

### REFERENCES

Sugawara, K., and Oyama, F. Fluorogenic Reaction and Specific Microdetermination of Ammonia (1981). J Biochem. 89, 771-774.
Corbin, J. Liquid Chromatographic-Fluorescence Determination of Ammonia from Nitrogenase Reactions: A 2-Min Assay. (1984). Applied and Environmental Microbiology, 1027-1030.

### Example 3

### PBEF/Nampt PNC1/OPT-Based Nicotinamide Assay

A sample protocol for measuring the activity of PBEF/Nampt is as follows:

### Reaction 1 Conversion of Nicotinamide by PBEF

Prepare a master-mix for the total number of reactions to be performed. For each individual reaction, mix the following in the order presented (the total volume for each reaction is 100 uL). Prepare two reactions for each condition to be assayed. The first reaction will serve as a blank to measure the initial fluorescence of the nicotinamide present (Fₒ). The second sample will measure the fluorescence of the residual nicotinamide present after reaction with PBEF (F₁). The difference in fluorescence between these two measurements (F₁-Fₒ) is equivalent to the quantity of fluorescence attributed to the amount of nicotinamide converted by PBEF (and is thus proportion to PBEF activity).

### Step 1)

Mix the following reagents in the order presented:
a) 10 uL 10× Assay Buffer (250 mM Tris-Cl [pH=8], 1.37 M NaCl, 27 mM KCl, 10 mM MgCl₂)
b) distilled/deionized H₂O (100 uL—volume of the other components)
c) An arbitrary concentration of Nicotinamide may be added (the suggested final concentrations for this assay are approximately 100-200 μM)
d) Purified PBEF/Nampt (˜2 ug)
*Any necessary inhibitors/activators may be added in here

For Reaction Fₒ, immediately proceed to step 3 to stop the reaction. For Reaction F₁, proceed on to step 2:

### Step 2)

Mix the reaction by pipetting, and subsequent gentle vortexing (setting 6 or 7). Incubate at 37° C. for the appropriate amount of time (typically 1 hour).

### Step 3)

Stop the reaction by heating the mixture at 95° C. for 5 minutes; alternatively, a chemical inhibitor of PBEF may be used.

### Reaction 2: Residual Nicotinamide→Ammonia

### Step 4)

Add 2 ug of a purified NAM-specific nicotinamidase. Mix the reaction by pipetting, and subsequent gentle vortexing (setting 6 or 7). Incubcate at room temperature for approximately 1 hour.
Reaction 3: Ammonia→Fluorescent adducts

### Step 5)

In some instances, when adding the OPT Developer it is advisable to work in a dark or dimly lit setting. For example, the lights can be dimmed prior to adding the developer to each reaction.
e) Add 100 uL of the OPT Developer Mix to each reaction.
f) Mix each sample by vortexing briefly for 5 s at the highest setting.
g) Immediately cover all of the samples with Aluminum foil, and incubate the reactions on an orbital shaker for 1 hour at room temperature. Once covered, ambient lighting may be restored.

### Step 6)

If necessary samples may be transferred to a 96-well plate for reading (do this under dim light). Read the fluorescence on a fluorometer with filters set to excitation (˜420 nm+/−20, and emission ˜450 nm (+/−20). A 0.1 s or 1 s read is generally recommended.
Subtract the value for Fₒ from F₁ for each reaction condition. ΔF (F₁-Fₒ) is proportional to the amount of nicotinamide converted by PBEF during the specified reaction time, and is thus an indicator of PBEF activity.

### Solutions

OPT Developer Reagent: A solution of 10 mM OPT and 10 mM DTT in 100% EtOH. Store at −20° C. in the dark until use. Cover with Aluminum foil in order to minimize exposure to light.

### Example 4

### Development of a Sirtuin Assay Kit

A representative kit was developed for conducting sirtuin assays. The assays can alternatively be run using a dual-enzyme coupled reaction (PNC1-GDH). Contents of the kit are described below. Sirtuin assays were carried out using the kits as described in the protocols below.

### A) Kit Contents

Kit contents are generally stored at −20° C. Modifications or variations in volumes or amounts of components described below are compatible with kits and assays described herein.
1) Assay Buffer: 2 tubes with 1 mL each of 10× Assay Buffer. Dilute to 1× with ddH20 prior to use.
2) Non-tagged Peptide Substrate: 1 tube with 140 uL of 5 mM H3K9ac substrate (TARK(ac)STG)) (SEQ ID NO:23). This is a sample substrate only; any substrate may be used in this assay.
3) β-NAD: a) 1 tube with 450 uL of 10 mM B-NAD, and b) 1 tube with 50 uL of 100 mM B-NAD. In some instances, it is preferable to keep β-NAD in concentrated aliquot form, and dilute it prior to use.
4) hSIRT1 Enzyme: 1 tube with 400 uL of 1.1 ug/uL full-length human SIRT1 enzyme.
5) yPNC1 Enzyme: 1 tube with 200 uL of 2 ug/uL yeast PNC1 enzyme
6) Developing Reagent: 2 vials with 10 mL each 1× Developer (10 mM OPT/10 mM DTT in 100% EtOH). Minimize exposure to light as outlined in the procedure.

### Additional Materials Provided:

1) 1 tube with 100 uL of ˜1 M Nicotinamide (NAM)
2) 1 tube with 5 uL of 100 mM SIRT1 Inhibitor (use at a final concentration of 10 uM in the reaction; the compound is dissolved in DMSO)

### Sirtuin Assay Protocols

In some embodiments, experiments are performed in duplicate or triplicate.

### Experiment 1: Nicotinamide Standard Curve

### 1) Prepare Nicotinamide Standards.

Prepare and label Eppendorf tubes corresponding to each final dose of Nicotinamide to be assayed (for example, 0, 2, 5, 10, 20, 40, 50 uM). Add the appropriate volume of Nicotinamide (for example, 1-3 uL) to each tube which will yield the desired NAM concentration following the addition of the Mastermix (the total volume after addition of the Mastermix will be 100 uL).

### 2) Prepare and Add Mastermix

Prepare a master-mix for the total number of reactions to be performed. One reaction will consist of the following (mixed in the order presented):
a) 10 uL 10×SIRT1 Assay Buffer (250 mM Tris-Cl [pH=8], 1.37 M NaCl, 27 mM KCl, 10 mM MgCl₂)
b) Distilled H₂O (100 uL—volume of the other components)
c) Purified yeast PNC1 enzyme (˜1 ug) ˜0.5-1 uL

Mix the master mix by pipetting, and subsequent gentle vortexing (setting 6 or 7), and add 100 uL of the mix to each reaction. Incubate at 37° C. for an hour.

### 3) Develop

In some instances, when adding the OPT Developer it is advisable to work in a dark or dimly lit setting. For example, the lights can be dimmed prior to adding the developer to each reaction.
h) Add 100 uL of the OPT Developer Mix to each reaction. Do this quickly to avoid lags in development time.
i) Mix each sample by vortexing briefly for 5 s at the highest setting.
j) Cover all of the samples with Aluminum foil, and incubate the reactions on an orbital shaker for 1 hour at room temperature. Once covered, ambient lighting may be restored.
k) Read the samples. Samples may be transferred to a 96-well plate for reading (do this under dim light). Read the fluorescence on a fluorometer with filters set to excitation ˜420 nm, and emission ˜460 nm.

For each sample value subtract off the blank standard (0 uM NAM), average values and construct a standard linear fit.

Experiment 2: Activity Titration with SIRT1 Inhibitor

### Controls

Blank Control: A No-NAD control reaction can be performed for each sample treatment. This does not need to be done in triplicate (once is usually sufficient). This reaction will have no Sirtuin activity (NAD is required), but will take into account auto-fluorescence from a drug (if any), auto-fluorescence from the peptide substrate (if any), and measurement background. β-NAD does not fluorescence in the assay up to a concentration of around 0.5 mM (the Km for NAD is around 160 uM).

For extremely high doses of β-NAD>0.5 mM, a No-Substrate reaction should be performed to correct for any NAD auto-fluorescence (usually only 10-20% of the signal).

A No-Enzyme control (less specific) can also be used.

1) Prepare Samples with Different Amounts of SIRT1 Inhibitor.
Prepare and label Eppendorf tubes corresponding to each final dose of Inhibitor to be assayed (for example, 0, 5, 10, 20 uM). Add the appropriate volume of Inhibitor (for example, 0.5-1 uL) to each tube which will yield the desired Inhibitor concentration following the addition of the Mastermix (the total volume after addition of the Mastermix will be 100 uL). Equalize volumes with DMSO.

### 2) Prepare and Add Mastermix

Prepare a master-mix for the total number of reactions to be performed. One reaction will consist of the following (mixed in the order presented):
a) 10 uL 10×SIRT1 Assay Buffer (250 mM Tris-Cl [pH=8], 1.37 M NaCl, 27 mM KCl, 10 mM MgCl₂)
b) Distilled H₂O (100 uL—volume of the other components)
c) 1 uL Non-tagged Substrate [5 mM stock, final concentration=50 uM (Km)]
*d) 1.5 uL β-NAD (10 mM stock, final concentration=150 uM (Km)]
   Do not add this to the No-NAD blank reactions.
e) Purified yeast PNC1 enzyme (˜1 ug) ˜0.5-1 uL
f) Purified human SIRT1 enzyme (˜1 ug) ˜1 uL

Mix the master mix by pipetting, and subsequent gentle vortexing (setting 6 or 7), and add 100 uL of the mix to each reaction. Incubate at 37° C. for an hour.

### 3) Develop

In some instances, when adding the OPT Developer it is advisable to work in a dark or dimly lit setting. For example, the lights can be dimmed prior to adding the developer to each reaction.
a) Add 100 uL of the OPT Developer Mix to each reaction. Do this quickly to avoid lags in development time.
b) Mix each sample by vortexing briefly for 5 s at the highest setting.
c) Cover all of the samples with Aluminum foil, and incubate the reactions on an orbital shaker for 1 hour at room temperature. Once covered, ambient lighting may be restored.
d) Read the samples. Samples may be transferred to a 96-well plate for reading (do this under dim light). Read the fluorescence on a fluorometer with filters set to excitation ˜420 nm, and emission ˜460 nm.

For each sample value subtract off the blank control reaction e.g. ([X_{uM}Drug]_{+NAD}−[X_{uM} Drug]_{-NAD}), average values, etc.

### Experiment 3: Km Determination of Substrate (Michaelis-Menten Kinetics)

### Controls

Blank Control: A No-NAD control reaction can be performed for each sample treatment (each different dose of substrate). This does not need to be done in triplicate (once is usually sufficient). This reaction will have no Sirtuin activity (NAD is required), but will take into account auto-fluorescence from the peptide substrate (if any), and measurement background. β-NAD does not fluorescence in the assay up to a concentration of around 0.5 mM (the Km for NAD is around 160 uM).

For extremely high doses of β-NAD>0.5 mM, a No-Substrate reaction should be performed to correct for any NAD auto-fluorescence (usually only 10-20% of the signal).

A No-Enzyme control (less specific) can also be used.

1) Prepare Samples with Different amounts of H3K9ac Substrate
Prepare and label Eppendorf tubes corresponding to each final dose of Substrate to be assayed (for example 0, 20, 40, 80, 200, 400 uM). Add the appropriate volume of substrate (for example 0-8 uL) to each tube which will yield the desired substrate concentration following the addition of the Mastermix (the total volume after addition of the Mastermix will be 100 uL). Equalize volumes with distilled water.

### 2) Prepare and Add Mastermix

Prepare a master-mix for the total number of reactions to be performed. One reaction will consist of the following (mixed in the order presented):
a) 10 uL 10×SIRT1 Assay Buffer (250 mM Tris-Cl [pH=8], 1.37 M NaCl, 27 mM KCl, 10 mM MgCl₂)
b) Distilled H₂O (100 uL—volume of the other components)
c) Non-tagged Substrate (already present in reaction tube)
*d) 1.5 uL β-NAD (10 mM stock, final concentration=150 uM (Km)]
   Do not add this to the No-NAD blank reactions.
e) Purified yeast PNC1 enzyme (˜1 ug) ˜0.5-1 uL
f) Purified human SIRT1 enzyme (˜1 ug) ˜1 uL

Mix the master mix by pipetting, and subsequent gentle vortexing (setting 6 or 7), and add 100 uL of the mix to each reaction. Incubate at 37° C. for an hour.

### 3) Develop

In some instances, when adding the OPT Developer it is advisable to work in a dark or dimly lit setting. For example, the lights can be dimmed prior to adding the developer to each reaction.
e) Add 100 uL of the OPT Developer Mix to each reaction. In some embodiments, it is preferable to do this quickly to avoid lags in development time.
f) Mix each sample by vortexing briefly for 5 s at the highest setting.
g) Cover all of the samples with Aluminum foil, and incubate the reactions on an orbital shaker for 1 hour at room temperature. Once covered, ambient lighting may be restored.
h) Read the samples. Samples may be transferred to a 96-well plate for reading (do this under dim light). Read the fluorescence on a fluorometer with filters set to excitation ˜420 nm, and emission ˜460 nm.

For each sample value subtract off the blank control reaction (e.g. [x_{uM}Substrate_{+NAD}]−[x_{uM}Substrate_{-NAD}] Average replicates and plot Fluorescence (y) versus substrate concentration (x). The plot may be fit to a M-M one-site saturation curve.

A standard curve may be used to convert fluorescence into amount of Nicotinamide produced.

### Example 5

### Assaying SIRT3 Activity

Using methods and materials described in Examples 1-4, assays developed herein were successfully used to measure SIRT3 activity on acetylated Acetyl CoA synthetase whole protein, and also with native peptides, indicating that assays described herein can be applied to all Sirtuin proteins.

Having thus described several aspects of at least one embodiment of this invention, it is to be appreciated various alterations, modifications, and improvements will readily occur to those skilled in the art. Such alterations, modifications, and improvements are intended to be part of this disclosure, and are intended to be within the spirit and scope of the invention. Accordingly, the foregoing description and drawings are by way of example only.

### EQUIVALENTS

Those skilled in the art will recognize, or be able to ascertain using no more than routine experimentation, many equivalents to the specific embodiments of the invention described herein. Such equivalents are intended to be encompassed by the following claims.

What is claimed is:

1. A method for measuring the activity of an enzyme, the method comprising:
combining the enzyme with β-nicotinamide adenine dinucleotide, and optionally an additional substrate, to form a reaction mixture, wherein the enzyme metabolizes β-nicotinamide adenine dinucleotide to produce nicotinamide,
adding to the reaction mixture a nicotinamidase in an amount sufficient to produce ammonia from the nicotinamide, and
detecting the amount of ammonia produced,
wherein the amount of ammonia produced is indicative of the activity of the enzyme.

2. The method of claim 1, wherein the enzyme is a Sirtuin.

3. The method of claim 2, wherein the Sirtuin is SIRT1.

4. The method of claim 2, wherein the additional substrate is an acetylated polypeptide.

5. The method of claim 1, wherein the enzyme is a glycohydrolase, optionally CD38, or wherein the enzyme is a mono or poly (ADP) ribosyltransferase (mART/PARP).

6. (canceled)

7. (canceled)

8. The method of claim 1, wherein the nicotinamidase is PNC1 or a homolog thereof.

9. The method of claim 1, wherein detection of ammonia comprises reaction of ammonia with o-phthalaldehyde and a reducing agent to produce a fluorescent product, and wherein the fluorescent product is detected.

10. The method of claim 9, wherein the reducing agent is DTT, β-mercaptoethanol, thioglycolic acid or sodium hydrosulfite.

11.-13. (canceled)

14. The method of claim 1, wherein the enzymatic reaction is terminated prior to addition of the nicotinamidase.

15. The method of claim 14, wherein detection of ammonia comprises addition of a-Ketoglutarate and NADPH and taking a first absorbance measurement, followed by addition of glutamate dehydrogenase and taking a second absorbance measurement, and wherein the difference in absorbance between the first and second absorbance measurements is indicative of the amount of ammonia present.

16. The method of claim 15, wherein the a-Ketoglutarate and NADPH are added simultaneously with the nicotinamidase, or wherein the a-Ketoglutarate and NADPH are added after the nicotinamidase.

17. (canceled)

18. The method of claim 1, wherein the method comprises a method for screening a test molecule for modulation of the activity of an enzyme wherein an increase in the amount of ammonia produced in the presence of the test molecule indicates that the test molecule is an activator of the enzyme, and wherein a decrease in the amount of ammonia produced in the presence of the test molecule indicates that the test molecule is an inhibitor of the enzyme.

19.-34. (canceled)

35. A method for measuring the amount of nicotinamide in a sample, the method comprising:
contacting a sample containing nicotinamide with a nicotinamidase in a reaction mixture, wherein the nicotinamidase produces ammonia from the nicotinamide, and
detecting the amount of ammonia produced,
wherein the amount of ammonia produced is indicative of the amount of nicotinamide in the sample.

36. The method of claim 35, wherein the sample is a water sample, a food sample, a tissue sample, a cell sample or a soil sample.

37.-40. (canceled)

41. The method of claim 35, wherein the nicotinamidase is PNC1 or a homolog thereof.

42. The method of claim 35, wherein detection of ammonia comprises reaction of ammonia with o-phthalaldehyde and a reducing agent to produce a fluorescent product, and wherein the fluorescent product is detected.

43. The method of claim 42, wherein the reducing agent is DTT, β-mercaptoethanol, thioglycolic acid or sodium hydrosulfite.

44.-46. (canceled)

47. The method of claim 35, wherein the enzymatic reaction is terminated prior to addition of the nicotinimidase, and optionally wherein detection of ammonia comprises addition of a-Ketoglutarate and NADPH and taking a first absorbance measurement, followed by addition of glutamate dehydrogenase and taking a second absorbance measurement, and wherein the difference in absorbance between the first and second absorbance measurements is indicative of the amount of ammonia present.

48. (canceled)

49. The method of claim 47, wherein the α-Ketoglutarate and NADPH are added simultaneously with the nicotinamidase or wherein the a-Ketoglutarate and NADPH are added after the nicotinamidase.

50.-130. (canceled)

131. A kit for measuring the activity of an enzyme, the kit comprising:
an enzyme, optionally a substrate molecule, β-nicotinamide adenine dinucleotide, a nicotinamidase protein, and instructions for use of components of the kit for measuring the activity of an enzyme.

132.-137. (canceled)
